(19) United States
(12) Patent Application Publication
(10) Pub. No.: US 2025/0129361 A1
(43) Pub. Date: Apr. 24, 2025

(54) METHOD FOR MASSIVELY-PARALLEL SCREENING OF APTAMER SWITCHES

(71) Applicants: CZ Biohub SF, LLC, San Francisco, CA (US); The Board of Trustees of The Leland Stanford Junior University, Stanford, CA (US)

(72) Inventors: Hyongsok Soh, Palo Alto, CA (US); Alexander M. Yoshikawa, Redwood City, CA (US); Alexandra E. Rangel, Santa Clara, CA (US); Amani Hariri, Stanford, CA (US)

(21) Appl. No.: 18/708,142

(22) PCT Filed: Nov. 8, 2022
(86) PCT No.: PCT/US2022/049285

§ 371 Date: May 7, 2024

Related U.S. Application Data
(60) Provisional application No. 63/278,996, filed on Nov. 12, 2021.

(51) Int. Cl. C12N 15/10; C12Q 1/25; C12Q 1/48; C12Q 1/6806; C12Q 1/6825; C12Q 1/6874; G01N 21/64

(52) U.S. Cl. CPC C12N 15/1072; C12Q 1/25; C12Q 1/485; C12Q 1/6806; C12Q 1/6825; C12Q 1/6874; G01N 21/6428; G01N 2021/6432; G01N 2333/9015; G01N 2333/91245

(57) ABSTRACT

Methods and compositions for identifying molecular switches are provided.

### CROSS-REFERENCE TO RELATED PATENT APPLICATIONS

The present patent application clams benefit of priority to U.S. Provisional Patent Application No. 63/278,996, filed Nov. 12, 2021, which is incorporated by reference for all purposes.

### FEDERALLY SPONSORED RESEARCH AND DEVELOPMENT

This invention was made with Government support under contracts OD025342 and GM129313 awarded by the National Institutes of Health. The Government has certain rights in the invention.

### BACKGROUND OF THE INVENTION

Nature has evolved a diverse toolbox of nucleic acid-based molecular switches-known as ‘riboswitches’—that enable living organisms to sense and respond to environmental stimuli. Riboswitches are complex folded RNA domains that control gene expression via allosteric structural changes triggered by binding to a specific ligand [Serganov et al., Cell 2013, 152 (1-2), 17-24; Mandal, M. and Breaker, R. R., Nature Reviews Molecular Cell Biology 2004, 5 (6), 451-463]. For example, the bacterial glycine riboswitch facilitates glycine breakdown by controlling the expression of three genes required for degradation in response to glycine binding [Wittmann, A. and Suess, B. FEBS Letters 2012, 586 (15), 2076-2083]. Similar target-responsive RNA- and DNA-based molecular switches have potential utility in a variety of technological applications, and researchers have engineered a number of synthetic nucleic acid-based constructs that mimic naturally-occurring riboswitches and undergo similar binding-induced conformational switching. In some cases, these are used to trigger the same kinds of gene-regulatory functions as occur in nature [Wittmann, A. and Suess, B. FEBS Letters 2012, 586 (15), 2076-2083 but engineered riboswitches are also commonly utilized as signaling molecules in biosensing applications [Thavarajah et al., ACS Synthetic Biology 2020, 9 (1), 10-18]. Such biosensors are typically based on aptamers that undergo a reversible structure-switching mechanism, which is then coupled to either an optical or electrochemical readout. These engineered nucleic acid molecular switches have been used for a wide range of applications such as imaging metabolite dynamics in living cells [Paige et al., Science 2012, 335 (6073), 1194; You et al., Proceedings of the National Academy of Sciences of the U.S. Pat. No. 2,015,112 (21), E2756-E2765], real-time monitoring of drug molecules in live animals [Mage et al., Nature Biomedical Engineering 2017, 1 (5), 1-10; Arroyo-Currás et al., Proceedings of the National Academy of Sciences of the U.S. Pat. No. 2,017,114 (4), 645-650], and targeted cancer therapy [Wang et al., ACS Nano 2012, 6 (6), 5070-5077; Prusty et al., Nature Communications 2018, 9 (1); Mo et al., Nature Communications 2014, 5].

However, it remains a challenge to generate novel aptamer switches, because most aptamers assume a stably folded structure and do not undergo a binding-induced conformation change. The majority of aptamer switches are therefore created via a post-selection engineering approach—for example, converting the aptamer into a molecular beacon, split-aptamer construct, or intramolecular strand-displacement reagent [Feagin et al., ACS Sensors 2018, 3 (9), 1611-1615]. However, these approaches all rely on rational design, and therefore require prior knowledge of the aptamer structure and entail careful balancing of thermodynamic states. Such detailed structural characterization has only been achieved for a relatively small number of aptamers, and most design efforts rely on in silico predictions of secondary structure. However, even the most advanced modeling software often fails to account for non-canonical base-pairing motifs, such as G-quadruplexes and pseudoknots, and such structural elements are often critical to the function of both natural riboswitches and synthetic aptamer switches [Afanasyeva et al., Biophysics and Physicobiology 2019, 16, 287-294]. These predicted structures also typically cannot capture the three-dimensional folding of aptamer switches, which can be highly relevant to target recognition and binding. As a consequence, the initial aptamer switch design effort is often followed by a time-consuming trial-and-error process in which multiple constructs are fabricated and evaluated. To overcome these obstacles, specialized methods have been developed for the direct screening of structure-switching aptamers, such as capture-SELEX [Nutiu, R. and Li, Y., Journal of the American Chemical Society 2003, 125 (16), 4771-4778; Yang et al., Methods 2016, 106, 58-65]. Here, a solid support is modified with a short complementary DNA helper strand that hybridizes to the aptamer library in the absence of target, and which enables partitioning of sequences that undergo target binding-induced dissociation from the solid support as a result of undergoing a conformational change. While this approach has proven successful, considerable effort is required to perform the selection, as evidenced by the relatively small number of aptamer switches in the literature [Zhao, Q. and Cheng, L., Analytical and Bioanalytical Chemistry 2013, 405 (25), 8233-8239 Zhang et al., Analytical Chemistry 2009, 81 (21), 8695-8701; Ma et al., Frontiers in Chemistry 2019, 7 (FEB), 1-10; Tok et al., Talanta 2010, 81 (1-2), 732-736; Moutsiopoulou et al., Analytical Chemistry 2020, 92 (11), 7393-7398; Sergelen et al., ACS Sensors 2017, 2 (7), 916-923; Ferguson et al., Science Translational Medicine 2013, 5 (213), 1-7; Giannetti et al., Sensors and Actuators Reports 2021, 3 (January), 100030; Wu et al., Automated Platform for High-Throughput Screening of Base-Modified Aptamers for Affinity and Specificity. 2020, 1-23].

### BRIEF SUMMARY OF THE INVENTION

In some embodiments, a method for screening for molecular switches for a target molecule is provided. In some embodiments, the method comprises
(a) providing at least 100 different potential molecular switches comprising a random sequence, each of the at least 100 different potential molecular switches separated from the other on a solid surface or solid support, wherein the potential molecular switches comprise a first nucleic acid linked to a first label and a second nucleic acid linked to a second label, wherein the first label and the second label generate a detectable signal that changes depending on the proximity of the labels to each other;
(b) measuring the detectable signal in the presence and absence of the target molecule, and
(c) identifying molecular switches from the potential molecular switches in which the detectable signal changes depending on the presence of the target molecule, thereby identifying molecular switches for the target molecule.

In some embodiments, the solid surface is a flow cell.

In some embodiments, the providing comprises:
(i) forming the first nucleic acids in a nucleotide sequencing reaction comprising synthesis-by-sequencing, wherein the 5′ end of the first nucleic acids is linked to the flow cell and comprise 5′-3′, a first flow cell primer binding site, the random sequence, an enzyme cleavage site and a second flow cell primer binding site, thereby generating a nucleotide sequencing read for each first nucleic acid,
(ii) for each first nucleic acid, recording the location and nucleotide sequence on the flow cell,
(iii) labelling the first nucleic acids;
(iv) annealing the second nucleic acid to a 5′ portion of the first nucleic acid that does not include the random sequence, wherein the second nucleic acid comprises a sequence that is a reverse complement of the 5′ portion of the first nucleic acid; and
(v) wherein the measuring occurs in the flow cell.

In some embodiments, the 5′ portion comprises the first flow cell primer binding site.

In some embodiments, the first nucleic acid comprises an anchor sequence between the first flow cell primer binding site and the random sequence and wherein the 5′ portion comprises the anchor sequence and the second nucleic acid comprises a reverse complement of the anchor sequence.

In some embodiments, the labelling comprises cleaving the enzyme cleavage site in the first nucleic acids with an enzyme to form a new 3′ end of the first nucleic acids and end labeling the new 3′ end with the first label. In some embodiments, the end labelling comprises contacting a terminal transferase or ligase to the new 3′ end in the presence of the first label.

In some embodiments, the first label is a fluorophore and the second label is a quencher. In some embodiments, the first label is a quencher and the second label is a fluorophore. In some embodiments, the first label is a donor fluorophore and the second label is an acceptor fluorophore. In some embodiments, first label is an acceptor fluorophore and the second label is a donor fluorophore.

In some embodiments, the plurality of partitions is at least 1000 partitions.

In some embodiments, the random sequence is 10-50 (e.g., 20-40, e.g., 25-35) contiguous nucleotides long.

In some embodiments, the anchor sequence is 5-500, e.g., 5-100, 10-50, 12-100, 15-50, or 20-30 contiguous nucleotides long.

In some embodiments, the second nucleic acid comprises an aptamer sequence with affinity for the target molecule. In some embodiments, the aptamer sequence is between the second label and the reverse complement of the anchor sequence In some embodiments, the switching nucleic acid strand comprises a linker sequence between the random sequence and the reverse complement of the anchor sequence. In some embodiments, the linker sequence is 1-10 (e.g., 4-6) contiguous nucleotides long. In some embodiments, the linker sequence is a homopolymer sequence. In some embodiments, the homopolymer is poly T.

In some embodiments, the method further comprises contacting a first nucleic acid/second nucleic acid combinations identified as a molecular switch in the identifying step to the target molecule and measuring a change detectable signal between the presence and absence of the target molecule.

In some embodiments, the first nucleic acids comprise 5′-3′ the anchor sequence, a stem sequence, the random sequence, a reverse complement of the stem sequence, and the enzyme cleavage site, wherein the stem sequence and the reverse complement of the stem sequence form a double stranded stem in the absence of the target, thereby bringing the first label in proximity to the second label. In some embodiments, prior to the providing (a), the method comprises enriching for polynucleotides that are molecular switches, wherein the enriching comprises,
(a) providing a plurality of a test nucleic acids having a 3′ and a 5′ end, wherein the test nucleic acid comprises (i) the random sequence and (i) a double stranded stem sequence comprising a double-stranded recognition sequence for a sequence-specific endonuclease and primer binding sequences that include at least part of the double-stranded recognition sequence or is closer to the 3′ and 5′ ends than the double-stranded recognition sequence;
(b) contacting the test nucleic acids with a target molecule and the endonuclease, wherein the endonuclease cleaves the double stranded stem sequence unless the target molecule triggers a conformational shift in the test nucleic acids to cause the stem sequence to disrupt the double stranded stem sequence; and
(c) selectively amplifying intact nucleic acids with primers that anneal to the primer binding sequences, thereby enriching for molecular switches that change conformation in the presence of the target molecule.

In some embodiments, the 3′ end of the test nucleic acids comprises one strand of a second restriction enzyme recognition sequence, and
the enriching further comprises:

(i) contacting the plurality of test nucleic acids with a single-stranded oligonucleotide comprising the reverse complement of the second restriction enzyme recognition sequence, wherein if the double stranded stem sequence is present then the one strand is not available to anneal to the single-stranded oligonucleotide and if the double stranded stem sequence is not present the one strand and the single-stranded oligonucleotide anneal for form the second restriction enzyme recognition sequence; and
(ii) contacting the plurality of test nucleic acids from (i) with the second restriction enzyme, thereby cleaving nucleic acids in which the one strand and the single-stranded oligonucleotide anneal, thereby enriching for nucleic acids that form a stem loop.

Also provided is a method for screening for molecular switches. In some embodiments, the method comprises,
(a) providing in a plurality of partitions:
an aptamer nucleic acid comprising:
a first label,
an aptamer sequence with binding specificity for a target molecule,
a first anchor molecule, and
a switching nucleic acid strand comprising:
a second label,
a switch domain sequence,
a second anchor molecule that binds to the first anchor molecule, and
a linker sequence between the switch domain sequence and the anchor sequence; and
wherein the first label and the second label generate a detectable signal that changes depending on the proximity of the labels to each other,
wherein the switch domain sequence of the switching nucleic acid strand is different between partitions, such that at least a majority of partitions contain unique switch domain sequences;
(b) binding the first and second anchor molecules in the partitions;
(c) measuring the detectable signal in the partitions in the presence and absence of the target molecule; and
(d) identifying partitions in which the detectable signal changes depending on the presence of the target molecule, thereby identifying partitions containing a switch domain/aptamer sequence combination that functions as a molecular switch for the target molecule.

In some embodiments, the first anchor molecule is an anchor sequence and the second anchor molecule is a reverse complement of the anchor sequence.

In some embodiments, (a) comprises providing in the plurality of partitions, the switching nucleic acid strand, wherein the switch domain sequence of the switching nucleic acid strand is different between partitions; and the method further comprises nucleotide sequencing the switching nucleic acid strands in the partitions and recording the location the sequences to their respective partitions; providing the aptamer nucleic acids in the partitions; and then performing the hybridizing, the measuring and the identifying.

In some embodiments, the switching nucleic acid strand has a 3′ end and the first label is linked to the 3′ end and the aptamer nucleic acid has a 5′ end and the second label is linked to the 5′ end.

In some embodiments, the anchor sequence is 5-500, e.g., 5-100, 10-50, 12-100, 15-50, or 20-30 contiguous nucleotides long.

In some embodiments, the linker sequence is 1-10 (e.g., 4-6) contiguous nucleotides long.

In some embodiments, the linker sequence is a homopolymer sequence. In some embodiments, the homopolymer is poly T (i.e., deoxythymidine).

In some embodiments, the first label is a fluorophore and the second label is a quencher.

In some embodiments, the first label is a quencher and the second label is a fluorophore.

In some embodiments, the first label is a donor fluorophore and the second label is an acceptor fluorophore.

In some embodiments, the first label is an acceptor fluorophore and the second label is a donor fluorophore.

In some embodiments, the plurality of partitions is at least 1000 partitions.

In some embodiments, the partitions are flow cells.

In some embodiments, the method further comprises contacting the switch domain/aptamer sequence combination that functions as a molecular switch to the target molecule and measuring a change detectable signal between the presence and absence of the target molecule.

Also provided is a method for enriching for molecular switches. In some embodiments, the method comprises:
(a) providing a plurality of a test nucleic acids having a 3′ and a 5′ end, wherein the test nucleic acid comprises (i) a random sequence and (i) a double stranded stem sequence comprising a double-stranded recognition sequence for a sequence-specific endonuclease and primer binding sequences that include at least part of the double-stranded recognition sequence or is closer to the 3′ and 5′ ends than the double-stranded recognition sequence;
(b) contacting the test nucleic acids with a target molecule and the endonuclease, wherein the endonuclease cleaves the double stranded stem sequence unless the target molecule triggers a conformational shift in the test nucleic acids to cause the stem sequence to disrupt the double stranded stem sequence; and
(c) selectively amplifying intact nucleic acids with primers that anneal to the primer binding sequences, thereby enriching for molecular switches that change conformation in the presence of the target molecule.

In some embodiments, the method further comprises contacting selective amplified intact nucleic acids, or a target-binding portion thereof, with the target molecule and measuring for a change of conformation of the amplified intact nucleic acids in response to binding of the target molecule.

In some embodiments, one or more nucleotides at the 3′ and 5′ ends are not complementary such that the 3′ and 5′ ends do not anneal. In some embodiments, the 3′ and 5′ ends each comprise at least 4-10 nucleotides that do not anneal.

In some embodiments, the test nucleic acids further comprise a linker sequence between the random sequence and the 3′ end. In some embodiments, the random sequence is 10-50 (e.g., 20-40, e.g., 25-35) nucleotides long. In some embodiments, the linker sequence is 3′ from the random sequence. In some embodiments, the linker sequence is a homopolymer. In some embodiments, the linker sequence is 1-10 (e.g., 4-6) nucleotides long. In some embodiments, the double stranded stem sequence is 10-14 nucleotides long with nucleotides on either end being non-complementary. In some embodiments, the double stranded stem sequence is 12 nucleotides long with nucleotides on either end being non-complementary.

In some embodiments, the method further comprises after the providing and before the contacting, enriching the plurality for nucleic acids that form the double stranded stem sequence. In some embodiments, the 3′ end of the test nucleic acids comprises one strand of a second restriction enzyme recognition sequence, and
the enriching comprises:
(i) contacting the plurality of test nucleic acids with a single-stranded oligonucleotide comprising the reverse complement of the second restriction enzyme recognition sequence, wherein if the double stranded stem sequence is present then the one strand is not available to anneal to the single-stranded oligonucleotide and if the double stranded stem sequence is not present the one strand and the single-stranded oligonucleotide anneal for form the second restriction enzyme recognition sequence; and
(ii) contacting the plurality of test nucleic acids from (i) with the second restriction enzyme, thereby cleaving nucleic acids in which the one strand and the single-stranded oligonucleotide anneal, thereby enriching for nucleic acids that form a stem loop.

In some embodiments, the second restriction enzyme is DdeI.

In some embodiments, the method further comprises
(d) contacting the amplified intact nucleic acids stem loop nucleic acids with the target molecule and the endonuclease, wherein the endonuclease cleaves the double stranded stem sequence unless the target molecule triggers a conformational shift in the stem loop nucleic acids to cause the stem sequence to disrupt the double stranded stem sequence; and
(e) selectively amplifying intact nucleic acids with primers that anneal to the primer binding sequences, thereby further selecting for molecular switches that change conformation in the presence of the target molecule, wherein steps (d) and (e) are optionally repeated 1, 2, 3, 4, 5 or more times to further enrich for molecular switches that change conformation in the presence of the target molecule.

### Definitions

As used herein the term “aptamer” or “aptamer sequence” refers to a nucleic acid having a specific binding affinity for a target, e.g., a target molecule, wherein such target is other than a polynucleotide that binds to the aptamer or aptamer sequence through Watson/Crick base pairing.

An aptamer can be selected from an in vitro selection, such as a bead-based selection with flow cytometry or a high-density aptamer array. Various aptamers are known and described in the art, see, e.g., International Patent Publication Nos. WO 2014068553 and WO 2016018934, and US Patent Publication No. US20120263651. In some embodiments, an aptamer can have between 5 and 175 nucleotides (e.g., between 10 and 175, between 20 and 175, between 40 and 175, between 60 and 175, between 80 and 175, between 100 and 175, between 120 and 175, between 140 and 175, between 160 and 175, between 170 and 175, between 5 and 170, between 5 and 160, between 5 and 140, between 5 and 120, between 5 and 100, between 5 and 80, between 5 and 60, between 5 and 40, between 5 and 20, between 5 and 10, 5, 10, 20, 30, 40, 50, 60, 70, 80, 90, 100, 110, 120, 130, 140, 150, 160, 170, or 175 nucleotides).

The terms “nucleic acid”, “nucleic acid sequence”, “nucleic acid molecule” and “polynucleotide” may be used interchangeably herein and refer to a polymeric form of nucleotides of any length, either deoxyribonucleotides or ribonucleotides, or analogs thereof, and may include naturally occurring nucleotides and/or modified (e.g., non-natural) nucleotides. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular. In some embodiments, nucleic acids (e.g., natural and non-natural nucleic acids) can be chemically modified with one or more organic small molecules, e.g., boronic acid modified nucleic acids. Examples of modified nucleic acids can be found in, but are not limited to, those in, e.g., Gordon et al., ACS Chem Biol. Oct. 12, 2019; Meek et al., Methods. 106:29-36, 2016; and Chen et al., Bioorg Med Chem Lett. 26 (16): 3958-62, 2016).

As used herein, the term “oligonucleotide” can refer to a polynucleotide chain, including but not limited to those less than 200 residues long, most typically between 15 and 100 nucleotides long, but also intended to encompass longer polynucleotide chains. Oligonucleotides can be single- or double-stranded.

As used herein, the term “molecular switch” refers to a probe molecule capable of binding a target molecule, wherein the binding of the target molecule causes a change in conformation of the molecular switch that is detectable. Non limiting examples of a molecular switch are aptamers, antibodies, peptides, or other molecules that change conformation upon binding to a target molecule. In some embodiments, the molecular switch has a first conformation when bound to the target and a second conformation when not bound to the target, wherein one or both of the first conformation and the second conformation provides a detectable signal. In some embodiments, a change from one conformation to a second conformation results in a change in optical signal. Conformation switching probes may be reversible or non-reversible.

The term “sequence” as used, for example, in the context of an aptamer sequence, a nucleic acid sequence or an amino acid sequence may refer to the primary structure, e.g., the order of monomeric subunits, e.g., nucleotides or amino acids, and/or to the molecule having the primary structure.

The terms “label” and “detectable label” may be used interchangeably herein to refer to a molecule capable of detection, including, but not limited to, radioactive isotopes, fluorophores, quantum dots, nanoparticles (e.g., fluorescent nanoparticles), chemiluminescers, chromophores, enzymes, enzyme substrates, enzyme cofactors, enzyme inhibitors, chromophores, dyes, metal ions, metal sols, ligands (e.g., biotin, avidin, streptavidin or haptens) and the like. Exemplary detectable moieties suitable for use as detectable labels include affinity tags and fluorescent proteins. “Optical reporters” as used herein can include, for example, fluorescent dyes or other fluorescent molecules as well as other molecules that can produce an optical signal (e.g., light) that can be transmitted through a waveguide.

As used herein, the term “fluorophore” refers to a compound, e.g., a small molecule or a protein, which when excited by exposure to a particular wavelength of light, emits light at a different wavelength. Fluorophores can be characterized in terms of their emission profile, or “color.” For example, green fluorophores (e.g., green fluorescent protein (GFP), Cy3, FITC, and Oregon Green) are generally characterized by their emission at wavelengths in the range of 510-550 nm. Red fluorophores (e.g., red fluorescent protein (RFP), Texas Red, Cy5, and tetramethylrhodamine) are generally characterized by their emission at wavelengths in the range of 590-690 nm.

As used herein, the term “quencher” refers to a compound that is capable of reducing or absorbing the emission from a fluorophore. Quenching may occur by any of several mechanisms, including fluorescence resonance energy transfer, photo-induced electron transfer, paramagnetic enhancement of intersystem crossing, Dexter exchange coupling, and excitation coupling, such as the formation of dark complexes. One example of a quencher is a dark quencher, which can absorb excitation energy from a fluorophore and dissipates the energy as heat. Another example of a quencher is a fluorescent quencher, which can absorb excitation energy from a fluorophore and reemit this energy as light.

As used herein, the term “target analyte” refers to a molecule that can be recognized and bound by the aptamer in the aptamer switch polynucleotide. A target analyte can be a small molecule (e.g., a small organic molecule), a protein, a peptide, or a nucleic acid (e.g., DNA or RNA).

A “stem” as used herein refers to a double stranded polynucleotide portion of a larger nucleic acid in which two single stranded portions of the same nucleic acid are capable of annealing because they are reverse complements of each other. The stem can form in some embodiments from the 5′ and 3′ end sequences of the same nucleic acid, or from a sequence 20, 15, or 10 nucleotides or closer to the 3′ and 5′ ends.

As used herein, the term “flow cell” refers to a vessel having a chamber where a reaction can be carried out, an inlet for delivering reagents to the chamber and an outlet for removing reagents from the chamber. In some embodiments the chamber is configured for detection of the reaction that occurs in the chamber. For example, the chamber can include one or more transparent surfaces allowing optical detection of biological specimens, optically labeled molecules, or the like in the chamber. Exemplary flow cells include, but are not limited to those used in a nucleic acid sequencing apparatus such as flow cells for the Genome Analyzer™, MiSeq™, NextSeq™ or HiSeq™ platforms commercialized by Illumina, Inc. (San Diego, Calif.); or for the SOLiD™ or Ion Torrent™ sequencing platform commercialized by Life Technologies (Carlsbad, Calif.). Exemplary flow cells and methods for their manufacture and use are also described, for example, in WO 2014/142841 A1; U.S. Pat. App. Pub. No. 2010/0111768 A1 and U.S. Pat. No. 8,951,781.

### BRIEF DESCRIPTION OF THE DRAWINGS

FIG. 1A-C: Overview of the ADS construct and the high-throughput screening process used to convert known aptamers to molecular switches FIG. 1A) Design of the fluorophore-labeled switching strand and quencher-labeled aptamer strand. FIG. 1B) Target-induced conformational changes in the ADS construct result in a change in distance between the fluorophore and quencher, providing an optical readout. FIG. 1C) Overview of the screening process. First, the switching strand library is sequenced on the flow-cell. Second, the ADS constructs are assembled on the surface of the flow-cell via addition of aptamer strands. Lastly, target-responsive molecular switches are identified by sequentially imaging the flow-cell in buffer alone and with the target molecule. Imaging data from each ADS construct cluster reveals the presence of switches for which target binding results in increased (signal-on) or decreased (signal-off) fluorescence.

FIG. 2A-C: Analysis of the 1,000 best-performing ADS sequences. FIG. 2A) Histogram of the frequency with which each base-position within the ATP aptamer was complementary to an SD sequence. This analysis was based only on the longest complementary region for each SD, with complementary regions of <3 nucleotides discarded. Nucleotides at each position in the aptamer are labeled above the histogram.

FIG. 2B) Histogram of Smith-Waterman similarity distances between the ATP aptamer and the reverse-complement of the top 1,000 SDs from our screen. FIG. 2C) Secondary structure of the ATP aptamer as previously discovered via NMR28. Boxed regions m1, m2, and m3 indicate segments complementary to the three recurring SD motifs that we identified.

FIG. 3A-C: Identification and characterization of ATP aptamer switches. FIG. 3A) Results from the high-throughput screen of switching domains for the ATP aptamer. Orange and blue bars respectively represent the cluster intensity in buffer and 500 μM ATP. Error bars represent the standard deviation of five measurements. FIG. 3B) Extracted images of individual ATP-responsive ADS clusters (red circle) on the MiSeq flow-cell from multiple buffer and ATP cycles. FIG. 3C) Validation of selected ADS constructs identified from the high-throughput screen. 50 nM of fluorophore- and quencher-labeled ADS construct was incubated with various concentrations of ATP and measured on a plate reader (n=4 replicates). A two independent binding site model was used to fit the raw data and normalize the binding signals between 0 and 1, as represented by the solid lines. Error bars represent the standard deviation of the measurements.

FIG. 4A-C: Analysis of the top 1,000 unique glucose SD sequences. FIG. 4A) Predicted secondary structure of the phenylboronic acid-modified glucose aptamer glu1min. Red bolded Ts denote location of modifications. The boxed region m1 indicates a motif that was highly recurrent among the sequence elements targeted by our top SDs. FIG. 4B) Histogram of the frequency with which each aptamer base-position was complementary to an SD. FIG. 4C) Histogram of Smith-Waterman similarity distances between the glucose aptamer and the reverse-complement of the top 1,000 SD sequences.

FIG. 5A-C. Identification and characterization of phenylboronic acid-modified aptamer switches for glucose. FIG. 5A) Analysis of the top four aptamer switch clusters identified in our flow-cell screen. Orange and blue bars represent cluster intensity in buffer and 10 mM glucose, respectively. Error bars represent the standard deviation of the measurements (n=4). FIG. 5B) Extracted images of clusters glu-1, -2, and -3 (red circles) on the MiSeq flow cell for both the buffer and glucose cycles. FIG. 5C) Validation of the glucose affinity of the four aptamers shown in panel A. 50 nM labeled ADS construct was incubated with various concentrations of glucose and the fluorescence signal was measured on a plate reader. The solid lines represent the fitted single binding site model. Error bars represent the standard deviation of three measurements.

FIG. 6. De novo isolation of aptamer switches for real-time measurement. Direct ISD-SELEX to enrich unimolecular aptamer switches. A double restriction enzyme approach is utilized to eliminate non-hairpin forming sequences as well as inactive switches during target incubation. In the first round only, DdeI is used to remove the 3′ biotin “B” of non-hairpin forming sequences so that stable hairpins can be selectively captured on SA beads. Next, BamHI is used to remove the primer binding sites of those sequences that do not present a conformational change upon target binding. These steps together allow for selective amplification of our active switches.

FIG. 7. High-throughput screening method to identify optically functional ISD switches (ISD screen). Clusters of the ISD library are synthesized on the flowcell surface, followed by assembly of the optical format to assess switching, and characterization of ON signal switching behavior.

FIG. 8A-B. Validation of glucose responsive ISD switches. 8A) Imager results of the ISD screen for the enriched glucose library for the top 7 switch candidates. The dark blue bars represent the cluster intensity in the presence of buffer and the light blue bars depict the intensity after incubation with 5 mM glucose. 8B) Plate reader validation of top glucose ISD candidates identified from the screen.

### DETAILED DESCRIPTION OF THE INVENTION

### Introduction

The inventors have identified methods for quickly and efficiently identifying molecular switches. Molecular switches are molecules with binding affinity for a target molecule and that changes signal depending on the presence of absence of the target molecule (i.e., whether the molecular switch binds the target molecule or not). The inventors have discovered methods for identifying molecular switches from polynucleotide libraries having random sequences, in contrast to previously methods that required, for example, a design of polynucleotide sequences with molecular switch activity. For example, in some aspects, the methods providing a plurality of physically-separated different potential molecular switches comprising a random sequence, wherein the potential molecular switches comprise a first nucleic acid linked to a first label and a second nucleic acid linked to a second label, wherein the first label and the second label generate a detectable signal that changes depending on the proximity of the labels to each other. Physical separation can be for example, separate clusters of library members on a solid surface such as a flow cell. By measuring signal of the detectable signal in the presence and absence of the target molecule, one can identify molecular switches from the potential molecular switches in which the detectable signal changes depending on the presence of the target molecule, thereby identifying molecular switches for the target molecule. In some aspects, the methods involve generating part or all of the molecular switch (e.g., at least the random portion) as part of nucleotide sequencing, which can involve for example bridge PCR or other sequencing-by-synthesis methods, thereby attaching the molecular switch or part thereof to a solid support (for example a flow cell). Thus the random portion will be sequenced and linked to a solid support and can then subsequently be assayed for signal with and without the target molecule, allowing for identification of the sequence of polynucleotides having molecular switch activity.

In some aspects, methods for identifying molecular switches from known aptamers are provided. The method, as described in detail below, involves providing an aptamer nucleic acid that comprises: a first label, an aptamer sequence with binding specificity for a target molecule, and a first anchor molecule and a library of switching nucleic acids that comprise a second label, a switch domain sequence which differs between library members, and can be random), and a second anchor molecule that binds to the first anchor molecule. In some embodiments, the anchor molecule and the molecule having affinity for the anchor molecule are two polynucleotide strands that anneal but in other embodiments can be non-polynucleotide molecules. In view of the affinity for the switching nucleic acid and the aptamer nucleic acid, the two strands will be brought in proximity, but the target molecule-specific interaction of the aptamer sequence and the switching domain comprising the random sequence will only occur for molecules where the random sequence is capable of acting as a molecular switch. The first and second labels are selected such that signal generated by the interaction of the two labels differs depending on a change in conformation of the aptamer sequence relative to the switching domain sequence. The library can then be screened for changes in signal between the presence and absence of the target molecule of the aptamer, allowing one to screen a large library of potential switching sequences for those that “switch” depending on the presence of the target molecule while anchored via the annealing of the anchor sequence. Once switching sequences are identified one can form a molecular switch by covalently or non-covalently linking the selected switching sequence with the aptamer sequence to form a molecular switch.

A second method is provided for identifying a molecular switch. In this second method, an aptamer sequence need not be identified previously. In this method, a library of stem-containing sequences are generated that comprise a non-stem portion comprising a random sequence and the stem portion comprising a restriction enzyme recognition sequence. The library can be contacted with the target molecule of interest. In some embodiments, the library can be enriched for members that form the stem in the absence of the target molecule (for example by selectively cleaving molecules that do not form the stem). For instance, some members of the library that bind the target molecule will change conformation such that the double stranded stem sequence is disassociated. The restriction enzyme is contacted to the library in these conditions, thereby cleaving the stem portion of library members that do not change conformation, leaving intact those that changed conformation in response to the presence of the target molecule. These intact library members can then be amplified or otherwise identified and selected. In some embodiments, the library of stem-containing sequences can initially be enriched for those nucleic acids that form a stem by including a second restriction enzyme recognition sequence on an end of the nucleic acid that can form an intact second restriction enzyme recognition sequence with a provided nucleic acid that can anneal only when the stem is not formed. By cleaving the nucleic acids with the second restriction enzyme, only intact stem nucleic acids will be retained (not cleaved) thereby enriching for those sequences where stems are formed.

### Forming Library of Random-Containing Sequences and Sequencing

In some embodiments, the library of potential molecular switches comprising a random sequence are sequenced (and in so doing linked to a solid support) and then subsequently assayed for molecular switching activity. For example, in some embodiments, the solid support is a flow cell and the library of potential molecular switches, or at least a portion thereof having the random sequence, are provided in the flow cells such that unique potential molecular switch sequences are in different flow cells. For example, in some embodiments, a majority of the partitions contain a unique potential molecular switch sequence. In embodiments in which primers complementary to a primer binding site at the 5′ end of the library member are attached to the flow cells, the library members can be annealed to the primers and subsequently be sequenced via sequencing-by synthesis.

Sequencing techniques, such as sequencing-by-synthesis techniques, are a particularly useful method for sequencing the library members while attaching the members to the flow cell. Sequencing-by-synthesis can be carried out as follows. To initiate a first sequencing-by-synthesis cycle, one or more labeled nucleotides, DNA polymerase, and sequencing-by-synthesis primers, as well as any other appropriate reagents, can be contacted with one or more features on a solid support (e.g. feature(s) where nucleic acid primers are attached to the solid support). Those features where sequencing-by-synthesis primer extension causes a labeled nucleotide to be incorporated can be detected. Optionally, the nucleotides can include a reversible termination moiety that terminates further primer extension once a nucleotide has been added to the sequencing-by-synthesis primer. For example, a nucleotide analog having a reversible terminator moiety can be added to a primer such that subsequent extension cannot occur until a deblocking agent is delivered to remove the moiety. Thus, for embodiments that use reversible termination, a deblocking reagent can be delivered to the solid support (before or after detection occurs). Washes can be carried out between the various delivery steps. The cycle can then be repeated n times to extend the primer by n nucleotides, thereby detecting a sequence of length n. This method thereby detects the nucleotide sequence of the library member on the flow cell and also attaches it to the flow cell (allowing for later manipulation and testing as a molecular switch). Exemplary sequencing-by-synthesis procedures, fluidic systems and detection platforms that can be readily adapted for use with a composition, apparatus or method of the present disclosure are described, for example, in Bentley et al., Nature 456:53-59 (2008), PCT Publ. Nos. WO 91/06678, WO 04/018497 or WO 07/123744; U.S. Pat. Nos. 7,057,026, 7,329,492, 7,211,414, 7,315,019 or 7,405,281, and US Pat. App. Publ. No. 2008/0108082. Thus, the nucleotide sequence and location of the various library members can be determined and recorded allowed for association of particular sequences with active switching sequences as identified by the methods herein.

By including an enzyme cleavage site near the 3′ end of the library members but 5′ of sequencing primer sequence that resides closer to the 3′ end, the library members can be prepared for 3′ end-labeling. Exemplary enzyme cleavage sites can include, but are not limited to, for example, a DdeI recognition sequence, with the respective enzyme (e.g., in this example, DdeI) provided to the flow cell to cleave the recognition sequence. Once cleaved, the newly formed 3′ end at the cleavage site can be end-labeled with a nucleotide comprising a label. This can be achieved, for example by providing a terminal transferase or ligase and a labeled nucleotide to end-label the cleaved library members. As detailed below, the labeled library members can then be screened for molecular switch activity in a variety of ways by testing the label signal in the presence and absence of a target molecule and a second nucleic acid comprising a second label, wherein the signal of the detectable signal changes depending on the proximity of the two labels.

The library of potential switching nucleic acids can vary as desired by the user. In some embodiments, the library has at least 102, 103, 104, 105, 106, or 107 different unique members.

Methods for Identifying Molecular Switches from Known Aptamers

As noted above, in some embodiments methods are provided for identifying molecular switches from known aptamers by contacting a library of switching nucleic acids (e.g., linked to surface) to an aptamer nucleic acid that bind to each other via an anchor molecule, that in some cases can be an anchor nucleic acid sequence that anneals to the switching nucleic acid via reverse complementary anchor sequences. In some embodiments, an aptamer nucleic acid comprises: a first label, an aptamer sequence with binding specificity for a target molecule, and a first anchor molecule and a switching nucleic acid strand comprises: a second label, a switch domain sequence which differs between library members, and can be random), and a second anchor molecule that binds to the first anchor molecule on the switching nucleic acid. The order provided above can be 3′-5′ or 5′-3′. In embodiments in which the first and second anchor molecules are reverse complementary nucleic acid sequences, the orientation of the anchor nucleic acid sequence and switching nucleic acid strand are selected to be in opposite orientation, allowing the two single stranded nucleic acids to partially anneal via the anchor sequences. In aspects in which the switching nucleic acid strand library members are linked via their 5′ ends to a solid support (e.g., a flow cell) then the order above for the switching nucleic acid strand can be for example 3′-5′: second label, switch domain sequence and second anchor molecule that binds to the first anchor molecule. In other embodiments, the second label can be located internally in the nucleic acid sequence rather than at an end nucleotide.

The random sequence of the switching nucleic acid strand will differ between library members as this is the sequence being screened for its ability to act as a molecular switch with the aptamer sequence. The random sequence can have for example 6-20 contiguous nucleotides, e.g., 8-12, e.g., 6, 7, 8, 9, 10, 11, 12, 13, 14, 15, 16, 17, 18, 19 or 20 nucleotides.

The anchor sequence should be sufficiently long such that the switching nucleic acid and the aptamer nucleic acids can anneal in the assays for the molecular switch activity. Exemplary anchor sequences are for example, 5-500, e.g., 5-100, 10-50, 12-100, 15-50, or 20-30 nucleotides (that are optionally contiguous) long.

The switching domain and the anchor sequence can be linked directly or they can be linked via a linker sequence. In some embodiments, the linker sequence is a homopolymer. Optionally the linker sequence is has 1-10 (e.g., 4-6) nucleotides, e.g., 3, 4, 5, 6, or 7 nucleotides. In some embodiments, when the linker is a homopolymeric polynucleotide, the homopolymeric polynucleotide can contain monomeric units of nucleotides (e.g., poly-thymine, poly-adenine, poly-guanine, poly-cytosine, or poly-uracil nucleotides). In particular embodiments, a homopolymeric polynucleotide contains poly-thymine nucleotides. In other embodiments, a linker can contain a mixture of two or more types nucleotides, i.e., a mixture of thymine and adenine nucleotides, a mixture of thymine and guanine nucleotides, a mixture of thymine and cytosine nucleotides, or a mixture of thymine, adenine, and guanine nucleotides.

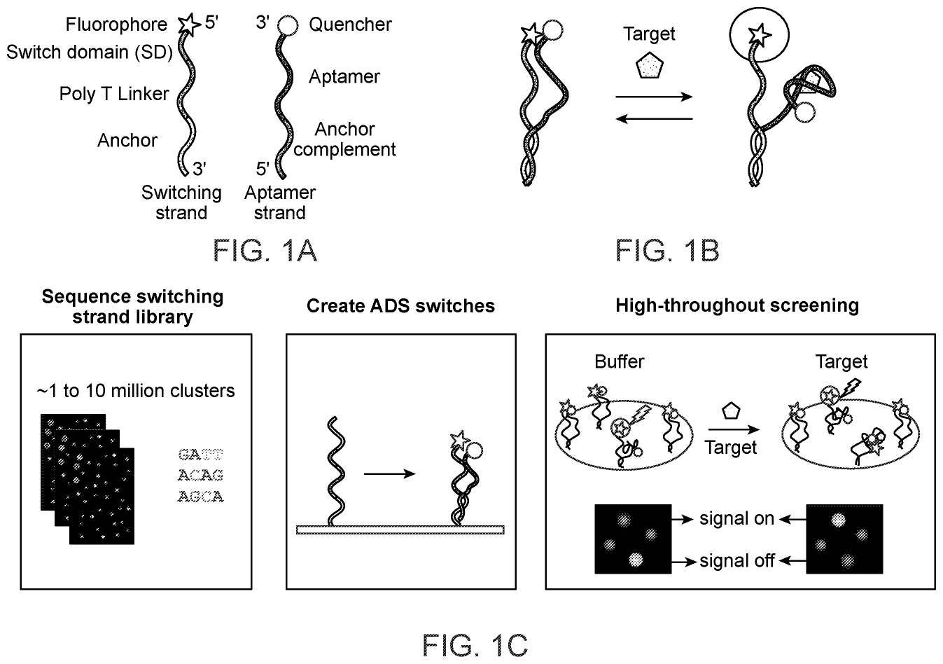

The second component, the aptamer-containing first nucleic acid sequence will comprise at least the aptamer sequence itself and a reverse complement of the anchor sequence on the switching nucleic acid or other binding molecule. An exemplary embodiment of this aspect is depicted in FIG. 1. It is believed any aptamer sequence known to bind a target molecule can be used as described herein. Exemplary aptamer structures are described in, e.g., Szostak, J. W. “In vitro selection of RNA molecules that bind specific ligands. Nature (1990); Gold L. et al., “Systematic evolution of ligands by exponential enrichment: RNA ligands to bacteriophage T4 DNA polymerase.” Science (1990); Jayasena, S. D. “Aptamers: an emerging class of molecules that rival antibodies in diagnostics.” Clin. Chem. (1999); and H. & van der Oost, J. Alternative affinity tools: more attractive than antibodies. Biochem. J. (2011).

In some embodiments, the aptamer can include a nucleic acid, a protein, a polymer comprising nucleic acids and proteins, or a chemically modified version thereof. The aptamer can be a synthetic polymer, e.g., a synthetic polymer comprising nucleic acids, proteins, and/or organic small molecules. A synthetic polymer can comprise natural and/or non-natural nucleic acids and natural and/or non-natural amino acids. In some embodiments, the natural and/or non-natural nucleic acids and natural and/or non-natural amino acids in the synthetic polymer can be further modified by one or more organic small molecules, e.g., a boronic acid modified uracil or other nucleotide.

In some embodiments, the aptamer can include a non-natural nucleotide. A non-natural nucleotide may contain a modification to either the base, sugar, or phosphate moiety compared to a naturally occurring nucleotide. A modification may be a chemical modification. Modifications may be, for example, of the 3′OH or 5′OH group of the backbone, of the sugar component, or of the nucleotide base. In some embodiments, the nucleotide is an unnatural nucleoside triphosphate. In some embodiments, one or more of the 4 naturally-occurring nucleotides (A, G, C, T/U) are replaced with a non-natural nucleotide. In some embodiments, two, three, or all four naturally-occurring nucleotides can be replaced by different non-natural nucleotides.

In some embodiments, a non-natural nucleotide may contain modifications to the nucleotide base. A modified base is a base other than the naturally occurring adenine, guanine, cytosine, thymine, or uracil. Examples of modified bases include, but are not limited to, C8-alkyne-uracil, uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifiuoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Examples of non-natural nucleotides include, but are not limited to, 5-substituted pyrimidines, 6-azapyrimidines and N-2 substituted purines, N-6 substituted purines, 0-6 substituted purines, 2-aminopropyladenine, 5-propynyluracil, 5-propynylcytosine, 5-methylcytosine, fluorinated nucleic acids, 5-substituted pyrimidines, 6-azapyrimidines and N-2, N-6 and O-6 substituted purines, including 2-aminopropyladenine, 5-propynyluracil and 5-propynylcytosine, 5-methylcytosine (5-me-C), 5-hydroxymethyl cytosine, xanthine, hypoxanthine, 2-aminoadenine, 6-methyl other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyl adenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine ([5,4-b][1,4]benzoxazin-2 (3H)-one), phenothiazine cytidine (1H-pyrimido[5,4-b][1,4]benzothiazin-2 (3H)-one), phenoxazine cytidine (e.g., 9-(2-aminoethoxy)-H-pyrimido[5,4-b][1,4]benzoxazin-2 (3H)-one), carbazole cytidine (2H-pyrimido[4,5-b]indol-2-one), pyridoindole cytidine (H-pyrido[3′,2′: 4,5]pyrrolo[2,3-d]pyrimidin-2-one), 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, and 2-amino-2′-deoxyadenosine. Examples of other synthetic nucleotides may be found in, e.g., Malyshev Nature. 509 (7500): 385, 2014.

The first and second labels can be selected such that they interact due to physical proximity and thus produce a change in detectable signal (which can be increased signal or decreased signal) as the two labels change from “close” to “far.” One non-limiting implementation includes optical reporters that comprise fluorophore donor/acceptor pairs that generate a fluorescent signal via Förster (or fluorescence) resonance energy transfer (FRET). The fluorescence intensity generated can in some embodiments be proportional to the amount of target bound to the aptamers, which enables quantitative measurements. FRET is a process by which radiationless transfer of energy occurs from an excited state fluorophore to a second chromophore in close proximity. The range over which the energy transfer can take place is limited to approximately 10 nanometers (100 angstroms), and the efficiency of transfer is extremely sensitive to the separation distance between fluorophores. Thus, as used herein, the term “FRET” refers to a physical phenomenon involving a donor fluorophore and a matching acceptor fluorophore selected so that the emission spectrum of the donor overlaps the excitation spectrum of the acceptor, and further selected so that when donor and acceptor are in close proximity (usually 10 nm or less), excitation of the donor will cause excitation of and emission from the acceptor, as some of the energy passes from donor to acceptor via a quantum coupling effect. Thus, a FRET signal serves as a proximity gauge of the donor and acceptor; only when they are in close proximity is a signal generated. The FRET donor moiety (e.g., donor fluorophore) and FRET acceptor moiety (e.g., acceptor fluorophore) are collectively referred to herein as a “FRET pair”. For example, in some embodiments, the molecular switch brings the donor and acceptor into close proximity upon binding of a target molecule but not when the target is absent. Alternatively, in some embodiments, the molecular switch brings the donor and acceptor into close proximity when the target molecule is absent and not upon binding of a target molecule. In some embodiments, the donor fluorophore and the acceptor fluorophore in a FRET pair are chosen such that the excitation wavelength of the donor fluorophore and the excitation wavelength of the acceptor fluorophore are sufficiently different from each other such that detection of one does not significantly (e.g., greater than 5 or 10%) affect detection of the other. This reduces false positive signals that might otherwise occur from detection of the two fluorophores. For example, the excitation wavelength of the one of the fluorophores can be in the blue wavelength range (e.g., between 450 nm and 495 nm, 450 nm and 490 nm, 450 nm and 480 nm, 450 nm and 470 nm, 450 nm and 460 nm, 460 nm and 495 nm, 470 nm and 495 nm, 480 nm and 495 nm, or 490 nm and 495 nm), and the excitation wavelength of the other fluorophore can be in the green wavelength range (e.g., between 500 nm and 570 nm, 500 nm and 560 nm, 500 nm and 550 nm, 500 nm and 540 nm, 500 nm and 530 nm, 500 nm and 520 nm, 500 nm and 510 nm, 510 nm and 570 nm, 520 nm and 570 nm, 530 nm and 570 nm, 540 nm and 570 nm, 550 nm and 570 nm, or 560 nm and 570 nm). Exemplary FRET donors include but are not limited to fluorescent dyes such as xanthene dyes (for example, Pvhodamine, fluorescein), naphthalimides, coumarins, cyanine dyes, oxazines, pyrenes, porphyrins, and acridines. Exemplary FRET pairs can be found in, for example, U.S. Pat. No. 8,124,357 and US Patent Publication 2018/0142222. Selection of FRET pairs is described in, e.g., Bajar et al., Sensors 2016, 16, 1488. Exemplary FRET acceptors can be a red-shifted dye or a dark quencher (i.e., a quencher that dissipates energy into heat). In yet other embodiments, the labels on the molecular switch can interact via static quenching or Dexter quenching.

In some embodiments, one of the first and second labels on the aptamer switch polynucleotide can be a fluorophore and the other of the first and second labels can be a quencher. In some embodiments, when a target analyte is not present, the hybridization between the displacement strand and the portion of the aptamer brings the fluorophore and quencher within proximity of each other such that the fluorescence from the fluorophore is quenched by the quencher. When a target analyte is present, the aptamer binds to the target analyte and does not hybridize to the displacement strand. As a result, the fluorophore and the quencher are not within quenching distance of each other and the fluorescence of the fluorophore can serve as a detectable readout for target analyte binding by the aptamer switch polynucleotide.

Examples of fluorophores, as well as quenchers, are known in the art, e.g., as described in Marras, Methods Mol Biol. 335:3-16, 2006; Kozma and Kele, Org Biomol Chem. 17 (2): 215-233, 2019; and Wang et al., Angew Chem Int Ed Engl. Mar. 7, 2019. Efficient and complete quenching of the fluorescence emitted from the fluorophore by the quencher depends in part on the overlap between the fluorophore emission and quencher absorption spectra. For example, fluorophore coumarin emits at emission wavelength around 472 nm and can be paired with quencher QSY35 which absorbs at wavelength around 475 nm. In another example, fluorophore Alexa 532 emits at emission wavelength around 554 nm and can be a paired with quencher QSY7 which absorbs at wavelength around 560 nm. In yet another example, fluorophore Alexa 647 emits at emission wavelength around 665 nm and can be paired with quencher QSY21 which absorbs at wavelength around 661 nm.

In other embodiments, a label can be a fluorophore whose fluorescence can be quenched when the displacement strand and the portion of the aptamer hybridize to each other in the absence of a target analyte. An example of such a fluorophore is 2-amino purine, whose fluorescence can be quenched when it is stacked with purines and/or pyrimidines (see, e.g., Jean and Hall, Proc Natl Acad Sci USA. 98 (1): 37-41, 2001).

In other embodiments, the labels in the aptamer switch polynucleotide can produce chemical and/or physical signals as a detectable readout when the aptamer switch polynucleotide binds to a target analyte. These signals can be monitored to infer binding to the target analyte. In one example, the labels can be electrochemical reporters (see, e.g., Ferguson et al., Sci Transl Med. 5 (213): 213ra165, 2013). A first label can be an electrode and a second label can be a redox reporter (e.g., methylene blue). Upon binding to the target analyte, the aptamer switch polynucleotide undergoes a conformational rearrangement that modulates the redox current and generates an electrochemical signal. Other chemical and/or physical signals or techniques that can be used to infer binding of the aptamer switch polynucleotide to a target analyte include, but are not limited to, anisotropy (see, e.g., Gokulrangan et al, Anal Chem. 77 (7): 1963-70, 2005; Chovelon et al., Biosensors and Bioelectronics 90:140-145, 2017), fluorescence polarization (see, e.g., Perrier et al., Biosensors and Bioelectronics 25:1652-1657, 2010), FETs (field-effect transistors) (see, e.g., Nakatsuka et al., Science 362:319-324, 2018), SERS (surface-enhanced Raman spectroscopy) (see, e.g., Chuong et al., Proc Natl Acad Sci USA. 114 (34): 9056, 2017; Sun et al., ACS Appl. Mater. Interfaces 8:5723-5728, 2016; and Lu et al., Analyst 139:3083, 2014).

Active molecular switches can be identified by measuring signal generated by the interaction of the first and second labels. Those members of the switching domain library comprising a random sequence capable of acting as a molecular switch in combination with the aptamer sequence will have a changed detectable signal depending on the presence and absence of the target molecule for the aptamer. Signal from library members can be linked to their nucleotide sequence by the location of the signal, thereby identifying random sequences that are able to act with the aptamers as molecular switches.

Once random sequences in the switching domain library have been identified, molecular switches can then be engineered by linking the aptamer and identified random sequence either covalently or non-covalently, optionally linked by a intervening linker, for form a molecular switch. For example in one embodiment, the identified random sequence and the aptamer sequence are parts of a single polypeptide strand that also comprises two labels as described herein such that the labels change proximity depending on whether the designed molecular switch is in the presence of absence of the target molecule.

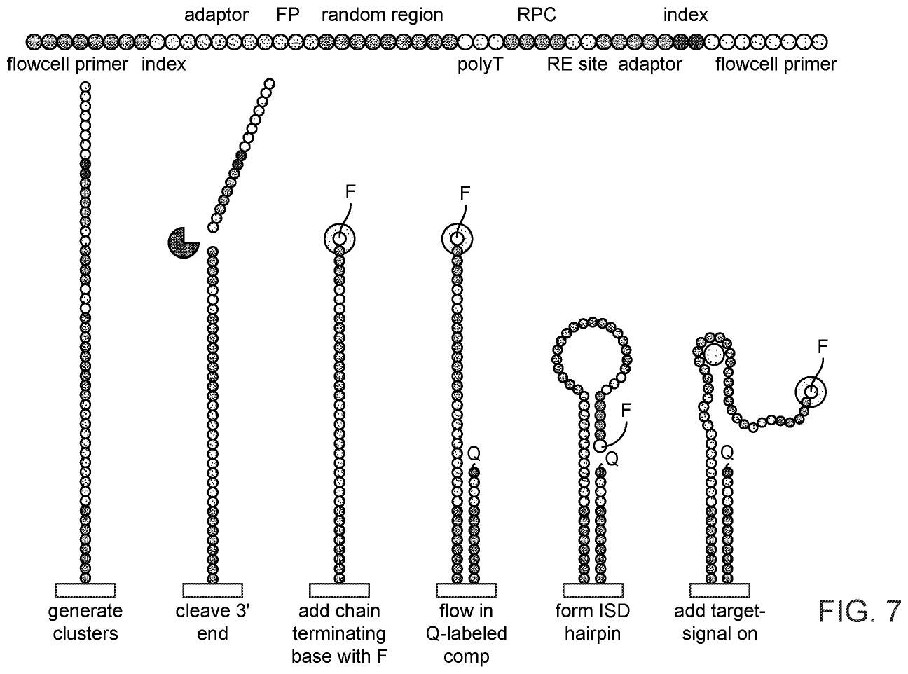

As noted above, in other embodiments, the methods need not start with a known aptamer sequence. In some embodiments, the methods rely on the formation of a stem (a double-stranded duplex) formed between two portions of a single nucleic acid. In these embodiments, the first nucleic acid comprises two sequences that anneal to form a stem, and that are separated by a random sequence and optionally other sequences such as a linker sequence. In these embodiments, one end (e.g., the 5′ end) of the first nucleic acids of the library is linked to a solid surface (e.g., a flow cell) and a label is included in the first nucleic acid (optionally at the opposite end, e.g., 3′ end) and then the second nucleic acid is hybridized to the first nucleic acid such that the second label on the second nucleic acid can interact with the first label when the first nucleic forms a stem, but the labels do not interact when the stem is absent (e.g., when in the presence of the target molecule triggers a conformational change in the first nucleic acid). An exemplary embodiment of this aspect is depicted in FIG. 7. As noted above, in this aspect, the first and second nucleic acids anneal, thereby bringing the two into proximity. In embodiments in which the first nucleic acid is linked to the solid surface at its 5′ end, the second nucleic acid will include a 3′ end portion that anneals to the 5′ end portion of the first nucleic acid. For instance, if the 5′ end sequence of the first nucleic acid comprises a first flow cell primer binding site the second nucleic acid can include at its 3′ end a complementary sequence to the first flow cell primer binding site. In some embodiments, the first label is on the 3′ end of the first nucleic acid and the second label is on the second nucleic acid and when the stem is formed in the first nucleic acid and the second nucleic acid is annealed to the first nucleic acid, the two labels are brought in proximity.

As described above, the entire library of first nucleic acids can be sequenced and labeled and then the second nucleic acid can be added and change of signal between the first and second label can be detected depending on the presence or absence of the target molecule. Again because the location and sequence of each library a member is known, one can identify those first nucleic acids that change configuration (disrupt the stem sequence) based on the presence of the target molecule.

Once first nucleic acids from the library have been identified for those that change configuration in response to the target molecule, molecular switches can be designed from these identified nucleic acid sequences. For example, molecular switches can be designed such that each strand of the stem sequence is end-labelled with labels as described herein whose combined signal changes in response to their proximity. Thus, once identified, molecular switches can be designed from the initial screening results.

As described in more detail below, one can, if desired, enrich (i) for nucleic acids that change structure in the presence of a target molecule such that the stem is disrupted in the presence of the target molecule or (ii) for nucleic acids that form a stem sequence in the absence of the target molecule or (iii) both (i) and (ii).

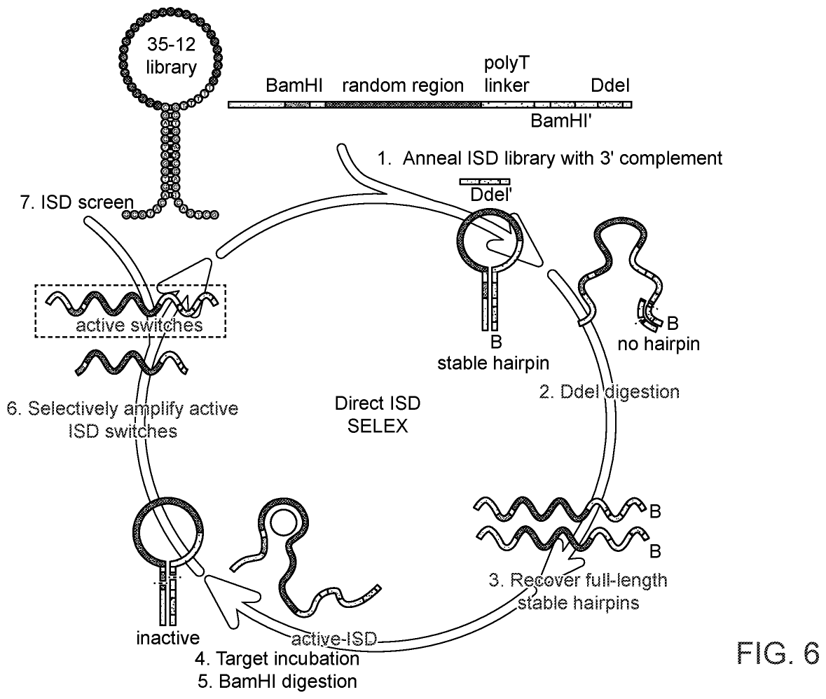

As demonstrated in the Examples, it is possible to perform an enrichment for library nucleic acids that do not form a stem in the presence of the target molecule. An aspect of this enrichment method is depicted in FIG. 6. Libraries of nucleic acids can be designed that have reverse complementary ends such that the ends would be expected to anneal and form a stem (double stranded portion formed by the two ends). A goal of the ultimate molecular switch screen is to identify nucleic acids for which the target molecule disrupts the stem (e.g., due to interaction of the random sequence with the target molecule). As disruption of the stem in the presence of the target molecule is one part of the screening methods described herein, it is beneficial to begin the screen with a library of nucleic acids that lack a stem in the presence of the target molecule, i.e., are more likely to meet the final criteria of the screen.

Thus, one can enrich for those library members that lack a stem in the presence of the target molecule. This can be achieved, for example, by generating nucleic acid library members that have reverse complementary end sequences that are predicted to form a stem sequence, where the stem sequence comprises a double-stranded restriction enzyme recognition sequence. This allows for initial selection of nucleic acid members in the presence of a target molecule by contacting the library members (e.g., in a bulk solution) with the restriction enzyme and then amplifying the nucleic acids with primers that are only present if the nucleic acid member is not cleaved by the restriction enzyme, i.e., if the stem sequence does not form a duplex and thus does not form the double-stranded restriction enzyme recognition sequence. Primer binding sites for amplifying intact (uncleaved) library members can be positioned closer to the 3′ and 5′ ends of the nucleic acids than the stem sequences. This can be achieved, for example, where one or more nucleotides at the 3′ and 5′ ends are not complementary such that the 3′ and 5′ ends do not anneal. For example, in some embodiments, the 3′ and 5′ ends each comprise at least 4-10 nucleotides that do not anneal (are not part of the stem) and comprise part or all of a primer binding site. Following contact of the library with the restriction enzyme, the library is amplified with primers that anneal to the primer binding sites, which remain on library members in which the stem was disrupted in the presence of the target molecule, but that are cleaved away from the remaining portion of the nucleic acids in members in which the stem remained intact in the presence of the target molecule. This method can include multiple (e.g., 2, 3, 4, 5, or more) rounds of contacting the library with the target molecule and restriction enzyme, followed by amplification, with each round further enriching for library members that remain intact following the restriction enzyme treatment. The restriction enzyme used can be any of a variety of restriction enzymes that cleave double stranded recognition sequences under the conditions used in the enrichment. Once enriched, the remaining library members can be screened for molecular switch activity as described herein (e.g., FIG. 7 or the library can be submitted to other types of enrichment, e.g., as described below.

Due to the various random intervening sequences between the two ends, not all nucleic acids will actually form a stem in the absence of a target molecule, for example due to interference from the random sequence or other intervening sequences. As formation of the stem is one part of the screening methods described herein, it can be beneficial to enrich the library for nucleic acids that form a stem in the absence of the target molecule. Methods for enriching for nucleic acids that form a stem in the absence of the target molecule are depicted for example in FIG. 6. For example, in these enrichment methods, the library can be designed to include one strand of a second restriction enzyme recognition sequence that overlaps with, but is not entirely encompassed by, the stem portion of the nucleic acids. For example, the stem sequence can include only 1, 2, 3, 4, 5, 6 or 7 but not all of the base pairs of the second restriction enzyme recognition sequence. Because the very end 3′ and 5′ nucleotides of the library nucleic acids are not complementary, the entire double-stranded second restriction enzyme recognition sequence is not formed by formation of the stem. In this case, if the nucleic acids form the stem, the second restriction enzyme recognition sequence is partially annealed to the reverse complementary sequence in the stem but the second restriction enzyme recognition sequence is not completely double-stranded and so cannot be cleaved by a restriction enzyme that recognizes the second restriction enzyme recognition sequence. However, if the nucleic acid does not form a stem, for example due to interference from the random sequence or other reasons, then the second restriction enzyme recognition sequence is available to anneal to other polynucleotides. By supplying an oligonucleotide complementary sequence to the strand of the second restriction enzyme recognition sequence on the library nucleic acids under conditions in which the oligonucleotide anneals, if the stem is not present, those library members that cannot form the stem will form an intact second restriction enzyme recognition sequence by annealing with the oligonucleotide. By contacting the library of nucleic acids with the oligonucleotide and the second restriction enzyme, member of the library that do not form a stem will be cleaved. Exemplary second restriction enzymes can include but are not limited to DdeI. By including an affinity tag at the 3′ end of the nucleic acids, one can enrich for intact nucleic acid members by selecting for those members that retain the affinity tag following contact with the second restriction enzyme. Exemplary affinity tags can include but are not limited to biotin. In embodiments in which biotin is the affinity tag, avidin or streptavidin (optionally linked to a solid support) can be used to bind intact members that include biotin while washing away cleaved members lacking the biotin, thereby enriching for intact members that form a stem in the absence of the target molecule. As with the method of enrichment for members that lack the stem in the presence of the target molecule, this enrichment method can be repeated in multiple (e.g., 2, 3, 4, 5, or more) rounds to further enrich for library members that form a stem in the absence of the target molecule.

### EXAMPLES

### Example 1

In this work, we describe a massively parallel screening strategy that could greatly accelerate the development of target-responsive molecular switches from existing aptamers without any a priori structural knowledge, eliminating the need for computer modeling or design heuristics. To achieve this, we built upon our recently developed non-natural aptamer array (N2A2) system, in which large numbers of natural DNA or base-modified aptamers are synthesized directly on the flow-cell of a modified Illumina MiSeq instrument and characterized in a massively parallel manner [Wu et al., Automated Platform for High-Throughput Screening of Base-Modified Aptamers for Affinity and Specificity. 2020, 1-23]. This allows us to rapidly screen as many as ˜1 million different switch scaffolds in a single experiment, rather than iterating through multiple cycles of design, synthesis and evaluation. The screening library consists of an array of anchored displacement strand (ADS) switch constructs, in which the aptamer of interest is coupled to a library of different switching strands with a variable ‘switch domain’ sequence. The screening process identifies those switching strand sequences that can efficiently hybridize to the aptamer in the presence of the target, but which become separated when target binding causes the aptamer sequence to assume a fully folded conformation.

We initially tested our approach with a natural DNA-based ATP aptamer and demonstrated the capacity to directly observe and identify thousands of fluorescent aptamer-based molecular switches. Interestingly, secondary structure prediction software failed to predict correct folding for many of the switches we discovered, highlighting the importance of non-canonical interactions to the structure and function of aptamer switches. We subsequently used the same strategy to identify aptamer switches containing chemically-modified bases. We first selected a novel boronic acid-modified aptamer that binds glucose, and then used our screening platform to create multiple high-affinity molecular switches that respond to glucose, far surpassing the sensitivity of previously reported glucose aptamers [Nakatsuka et al., Science 2018, 6750 (September), 1-11,]. Importantly, these switches exhibited a minimal decrease in affinity compared to the parent aptamer, and to our knowledge, represent the first example of a base-modified aptamer switch. These results demonstrate that our platform should offer a generalizable strategy for converting aptamers to target-specific molecular switches.

### Results and Discussion

### Strategy for High-Throughput Selection of Aptamer Switching Domains

Our screen draws inspiration from competition-based aptamer switch designs [Wilson et al., Nature Communications 2019, 10 (1), 1-9, however, in our strategy the aptamer and displacement strand are coupled together via base-pairing to form an ADS construct (FIG. 1A). The ‘aptamer strand’ consists of a known aptamer of interest, which is labeled at its 3′ end with a fluorescence quencher group and flanked at its 5′ end with an anchor sequence. The ‘switching strand’ is the variable component of the screening process, and is responsible for endowing the construct with the ability to undergo target binding-induced conformational switching. The switching strand comprises a sequence complementary to the aptamer strand's anchor sequence, a poly T linker, and a randomized 10-nucleotide switch domain (SD) sequence, and is fluorescently labeled at its 5′ end.

FIG. 1: Overview of the ADS construct and the high-throughput screening process used to convert known aptamers to molecular switches A) Design of the fluorophore-labeled switching strand and quencher-labeled aptamer strand. B) Target-induced conformational changes in the ADS construct result in a change in distance between the fluorophore and quencher, providing an optical readout. C) Overview of the screening process. First, the switching strand library is sequenced on the flow-cell. Second, the ADS constructs are assembled on the surface of the flow-cell via addition of aptamer strands. Lastly, target-responsive molecular switches are identified by sequentially imaging the flow-cell in buffer alone and with the target molecule. Imaging data from each ADS construct cluster reveals the presence of switches for which target binding results in increased (signal-on) or decreased (signal-off) fluorescence.

In a switching-compatible ADS construct, the complementary anchor regions of the two strands are hybridized, and the SD sequence also interacts with the aptamer sequence in the absence of target. Target binding causes the aptamer strand to undergo a conformational switch that changes the average distance between the fluorophore and quencher, resulting in altered fluorescent signal (FIG. 1B). In some cases, this conformational change will produce a ‘signal-on’ readout with increased fluorescence from target binding, whereas other constructs will undergo a ‘signal-off’ switching where fluorescence is more strongly quenched. Our approach bypasses the time-consuming and expensive process of structural analysis, rational design, and optimization by simply screening nearly every possible N10 SD sequence (˜1 million) in a single assay. This makes it possible to evaluate the entire sequence space and identify optimal switch constructs without requiring prior knowledge of the structure or binding sites of the aptamer.

The entire ADS screening process takes place on a MiSeq flow-cell, and involves three steps: 1) sequencing of the switching strand library, 2) assembly of the ADS aptamer constructs, and 3) identification of target-responsive aptamer switches (FIG. 1C). This allows us to directly link the genotype of each switching strand sequence to a functional phenotype (i.e., switching behavior) for the resulting ADS construct. In the first step, the switching strand library with a variable N10 SD region is sequenced on the MiSeq using standard Illumina sequencing protocols. We then assemble the ADS constructs on the surface of the flow-cell. Briefly, this entails targeted cleavage of the sequencing primer-complementary sequence adjacent to the randomized SD domain via a DdeI restriction enzyme site incorporated into the library molecules, after which the library clusters are fluorescently labeled with Cy3 using a terminal deoxynucleotidyl transferase (TdT) enzyme (see Methods for a detailed description of this step). The ADS constructs are then fully assembled by annealing the quencher-tagged aptamer strands onto the switching strand clusters. In the third and final step of the screening process, the flow-cell is washed with buffer and a fluorescent image of the flow-cell is acquired. The target molecule is then injected onto the flow-cell, and another fluorescent image is taken. By comparing the intensity of these two images, we can identify ADS constructs that undergo a target-induced change in fluorescent signal, whether signal-on or signal-off. The flow-cell is then washed with 50 mM NaOH to remove the target and aptamer from the ADS library clusters, after which the aptamer strand is re-annealed and replicate measurements are performed to identify outliers and characterize the variance in the experiment.

### Screening ATP-Responsive ADS Constructs

As an initial test of our high-throughput ADS screening platform, we used a well-studied DNA aptamer that binds ATP with a dissociation constant (K_{D}) of 6 μM [Serganov et al., Cell 2013, 152 (1-2), 17-24; Huizenga, D. E. and Szostak, J. W., Biochemistry 1995, 34 (2), 656-665] (Table S1). This aptamer has been extensively studied, and unlike most DNA aptamers, its three-dimensional structure has been solved via NMR studies [Lin, C. H. and Pate, D. J., Structural Basis of DNA Folding and Recognition in an AMP-DNA Aptamer Complex: Distinct Architectures but Common Recognition Motifs for DNA and RNA Aptamers Complexed to AMP]. Before proceeding with screening, we first validated the performance of the enzymatic steps involved with ADS synthesis on magnetic beads via flow cytometry (see Methods for details). These results confirmed successful enzymatic cleavage of primer-binding regions and successful enzymatic coupling of the fluorescent label. We then proceeded to set up the high-throughput ADS screen on our N2A2 platform using the ATP aptamer. After sequencing the switching strands, the ADS constructs were assembled on the surface of the flow-cell. Examination of the flow-cell images confirmed that the switching strands were selectively labeled with Cy3. The quencher-labeled aptamer strands were then hybridized onto these clusters, completing the formation of the ADS construct. The clusters were imaged in alternating cycles of buffer and 500 μM ATP, with five of each cycle performed in total. The cluster intensities calculated by the MiSeq software were linked to each cluster using a custom Python script. To eliminate false positives and reduce noise, clusters with signal below 100 RFU and above 1,000 RFU were filtered and discarded. We have found that clusters with low initial fluorescence are noisy and that clusters with intensities >1,000 RFU are typically caused by inaccurate image processing or by unwanted particles, such as dust, on the flow-cell. To eliminate data with high levels of variance, we also excluded clusters that had over 30% relative standard deviation (RSD) between either replicate buffer cycles or replicate ATP cycles. Finally, the switching domain clusters were sorted by the ratio of the signal in ATP compared to buffer.

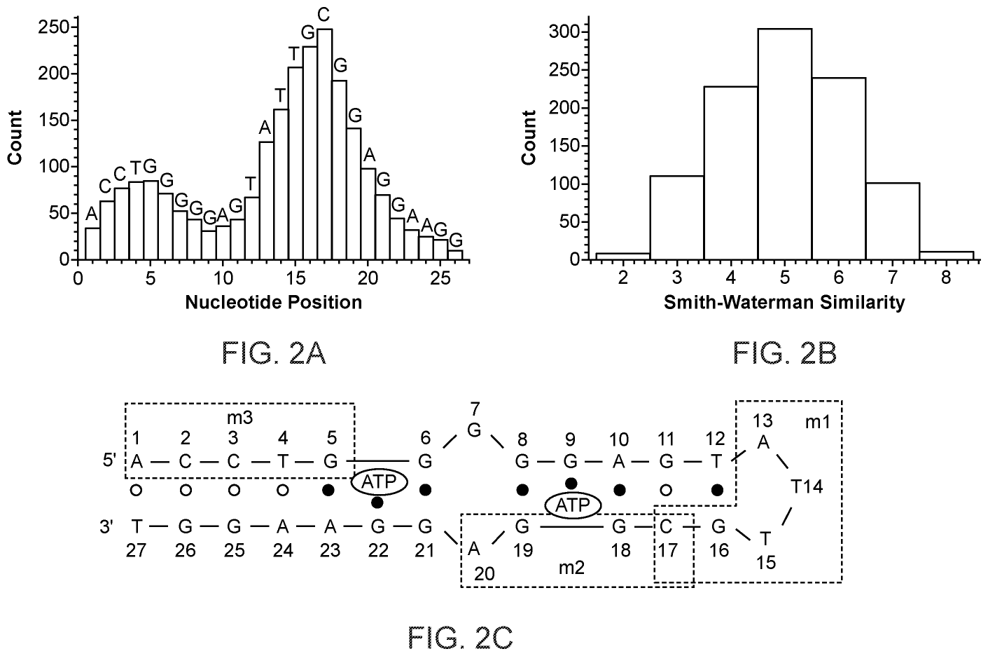

Analysis of the filtered data revealed thousands of ATP-responsive aptamer switches. While the screen can identify both signal-on and signal-off switches, the ATP aptamer yielded primarily signal-on aptamer switches. Of the ˜455,000 clusters, over 8,000 exhibited at least a two-fold change in fluorescence intensity after the addition of ATP. The top 1,000 unique SD sequences showed a more than four-fold increase in fluorescent intensity (Figure S5), with an eight-fold increase in intensity from the top-performing switch. To investigate whether particular regions of the ATP aptamer were preferentially interacting with SD regions in these switches, we created a histogram of the frequency with which each base-position in the ATP aptamer ended up being recognized by an SD sequence in the top 1,000 ADS switch candidates (FIG. 2A). We observed two clear peaks within the ATP aptamer sequence that were preferentially targeted for base-pairing by SD sequences. To our surprise, this sequence alignment analysis revealed that many of these SD regions had minimal complementarity with the aptamer (FIG. 2B). Indeed, more than 10% of the top 1,000 SD regions had a Smith-Waterman similarity score of 3 or less to the reverse-complement of the aptamer, meaning that only 3 of the 10 bases in the SD would be predicted to hybridize to the ATP aptamer. This leads us to believe that that many of these constructs must rely on extensive mismatch base-pairing or non-canonical nucleic acid interactions, which is in keeping with previous findings that naturally evolved riboswitches also extensively utilize such non-canonical interactions. [Serganov et al., Cell 2013, 152 (1-2), 17-24].

FIG. 2: Analysis of the 1,000 best-performing ADS sequences. A) Histogram of the frequency with which each base-position within the ATP aptamer was complementary to an SD sequence. This analysis was based only on the longest complementary region for each SD, with complementary regions of <3 nucleotides discarded. Nucleotides at each position in the aptamer are labeled above the histogram. B) Histogram of Smith-Waterman similarity distances between the ATP aptamer and the reverse-complement of the top 1,000 SDs from our screen. C) Secondary structure of the ATP aptamer as previously discovered via NMR [Lin, C. H. and Pate, D. J., Structural Basis of DNA Folding and Recognition in an AMP-DNA Aptamer Complex: Distinct Architectures but Common Recognition Motifs for DNA and RNA Aptamers Complexed to AMP]. Boxed regions m1, m2, and m3 indicate segments complementary to the three recurring SD motifs that we identified.

We determined that the two regions of the ATP aptamer that were preferentially targeted by the switching domains corresponded to distinct recurring sequence motifs by analyzing the top sequences using the motif discovery tool in the MEME suite (version 5.3.3) [Bailey et al., Nucleic Acids Research 2009, 37 (SUPPL. 2), 202-208]. We found three sequence motifs (m1, m2, and m3) that exhibited a statistically significant association with switching function (E-value <0.05). All three were complementary to different regions of the ATP aptamer (FIG. 2C) and overlapped one of the two regions of the aptamer identified in FIG. 2A. Motifs m1, m2, and m3 were respectively represented in 28.2%, 9.3%, and 4.9% of the top 1,000 sequences. By analyzing the secondary structure of the ATP aptamer as determined by NMR [Lin, C. H. and Pate, D. J., Structural Basis of DNA Folding and Recognition in an AMP-DNA Aptamer Complex: Distinct Architectures but Common Recognition Motifs for DNA and RNA Aptamers Complexed to AMP], we found that the m2 motif was complementary to one of the ATP binding pockets, whereas the m3 motif recognizes the stem of the aptamer—both representing regions that one might target with a rationally-designed SD. Interestingly, the m2 motif contained a mismatch in the SD sequence, indicating that imperfectly complementary displacement strands may yield optimal performance in some switch constructs. This is not surprising, given that mismatches can finely tune the thermodynamics of hybridization reactions. However, rationally designed displacement strands typically do not contain mismatches, and it is therefore likely that this subset of sequences would have been overlooked with such an approach. We were also surprised to see that the m1 motif was so abundantly represented—being present in more than a quarter of the top sequences—and this indicates that the short DNA loop recognized by m1 may be a particularly responsive target for the development of displacement strand-based switches. This is contrary to conventional wisdom, wherein such strands would typically be designed to target the binding pocket or the stem that stabilizes the aptamer structure in order to ensure that the aptamer cannot bind the target without first decoupling from the displacement strand. These results highlight the value that can be derived by employing a broader and more agnostic approach to identify displacement strands that confer optimal switching properties, rather than relying on potentially misleading a priori assumptions about structure-function relationships.

### Experimental Validation of ATP Aptamer Switches

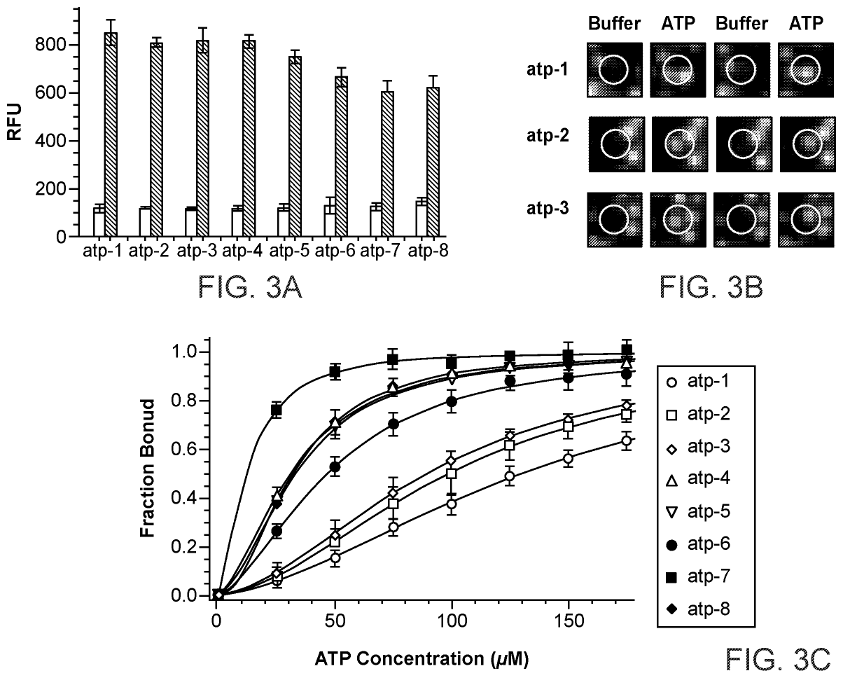

To verify the function of the aptamer switches, we chose a subset of eight individual ADS constructs for characterization and further analysis (Table S2). All eight sequences displayed a large—ranging from 6- to 8-fold—increase in fluorescence intensity when bound to ATP during the ADS screen, indicating a robust signal-on response (FIG. 3A). We chose the four that displayed the greatest percent change between the buffer and target cycles (atp-1-4), as well as the top-performing sequence with each of the three SD motifs identified above. Atp-4 was also the top sequence containing the m-3 motif, whereas atp-5 and -6 were respectively the best-performing sequences containing the m-1 and m-2 motifs. Finally, we selected two sequences (atp-7 and -8) that displayed low complementarity (three nucleotides) with the aptamer, but nevertheless exhibited a strong fluorescence increase in response to ATP binding. Since the screen relies on cluster intensity values that are automatically extracted from the raw images by the MiSeq software, we further validated that these were true-positive responses by manually inspecting the original cluster images from the flow-cell using a custom Python script (FIG. 3B).

FIG. 3: Identification and characterization of ATP aptamer switches. A) Results from the high-throughput screen of switching domains for the ATP aptamer. Orange and blue bars respectively represent the cluster intensity in buffer and 500 μM ATP. Error bars represent the standard deviation of five measurements. B) Extracted images of individual ATP-responsive ADS clusters (red circle) on the MiSeq flow-cell from multiple buffer and ATP cycles. C) Validation of selected ADS constructs identified from the high-throughput screen. 50 nM of fluorophore- and quencher-labeled ADS construct was incubated with various concentrations of ATP and measured on a plate reader (n=4 replicates). A two independent binding site model was used to fit the raw data and normalize the binding signals between 0 and 1, as represented by the solid lines. Error bars represent the standard deviation of the measurements.

In order to confirm the activity of the aptamer switches, we chemically synthesized the selected switching strands and assembled fluorophore- and quencher-modified ADS constructs by annealing the switching and aptamer strands at a 1:1 ratio. We then measured the target-induced fluorescence response of a fixed concentration (50 nM) of these ADS constructs on a plate reader after titrating with various concentrations of ATP (FIG. 3C). Each construct displayed a dose-dependent fluorescence increase, in agreement with the results from the high-throughput screen, and we determined their K_{D} by fitting to a two-binding site model that has been previously used to characterize ISD constructs developed from the same aptamer. K_{D} values ranged from 12-157 μM, which is reasonable given that the original ATP aptamer has a K_{D} of 6 μM. It is worth noting that the highest-affinity construct we tested was atp-7, which was chosen based on its low degree of complementarity to the ATP aptamer—this again highlights the fact that the determinants of an effective SD for an aptamer switch might be somewhat counter-intuitive based on current design principles.

We next sought to analyze the folding of the best switches by computationally modeling with three popular nucleic acid structure-prediction tools: NuPack [Zadeh et al., Journal of computational chemistry 2009, 32 (1), 170-173], UNAFold [Markham, N. R. and Zuker, M., Bioinformatics: Structure, Function and Applications; Keith, J. M., Ed.; Humana Press: Totowa, NJ, 2008; pp 3-31], and RNAFold [Gruber et al., Nucleic acids research 2008, 36 (Web Server issue), 70-74]. However, these different packages failed to provide consistent results, and in most cases predicted very dissimilar structures. It has been previously established that these different structure prediction packages can provide different structures for an aptamer, and that the predicted structures are often not reflective of the aptamer's true structure [Afanasyeva et al., Biophysics and Physicobiology 2019, 16, 287-294]. This is particularly true when the secondary structure is influenced by non-canonical interactions. In fact, NuPack and UNAFold often predicted no interaction between switching and aptamer strands apart from the anchor region. The structures predicted by RNAFold all displayed some mechanism by which the SD could interact with the aptamer, where the predicted structures were heavily stabilized by mismatched base-pairs. Although it is outside the scope of this work to determine the exact structure for these particular switches, the fact that we cannot obtain consistent predictions for our best performing scaffolds underscores the challenges of designing optimal switches using computationally-assisted rational design strategies. But by covering the full range of available sequence space for the SD domain, our screen can rapidly identify promising aptamer switch constructs that would likely be otherwise overlooked.

### A Novel Base-Modified DNA Aptamer Switch for Glucose

We next explored whether our method could be generalized to aptamer sequences containing non-natural, chemically-modified nucleic acids in order to identify high-affinity aptamer switches responsive to glucose. Small-molecule targets such as glucose have proven to be challenging targets for natural DNA and RNA aptamers—in part because of the limited number of chemical functional groups they offer as recognition sites [Meek et al., Methods 2016, 106, 29-36]. Our group and others have previously demonstrated that aptamers that incorporate chemically-modified nucleobases can achieve robust binding to such challenging targets [Yoshikawa et al., Discovery of Indole-Modified Aptamers for Highly Specific Recognition of Protein Glycoforms, bioRxiv 2021], and we therefore set out to isolate such an aptamer as the foundation for a glucose-responsive switch [Huizenga, D. E. and Szostak, J. W., Biochemistry 1995, 34 (2), 656-665].

To this end, we first employed our previously published Click-PD screening method [Gordon et al., ACS Chemical Biology 2019, 14 (12), 2652-2662 to generate a boronic acid-modified aptamer that achieves high affinity for glucose. Briefly, click chemistry is used to covalently couple alkyne-labeled chemical modifications onto libraries of monoclonal aptamer particles displaying sequences that incorporate azide-tagged non-natural uracil nucleotides. Fluorescence-activated cell sorting (FACS) is then used to interrogate the binding of these base-modified aptamer particles to a labeled target, and those that exhibit high fluorescence—and thus high affinity—can be individually sorted in a high-throughput manner. We chose phenylboronic acid as the base modification for this selection, as it forms stable cyclic boronate esters with saccharide diols, and should therefore facilitate the generation of high affinity aptamers to glucose. We also appended an ortho-aminoethyl moiety onto the phenylboronic acid modification used in our selection, which has been shown to further facilitate aptamer binding to saccharides at physiological pH [Collins et al., Journal of Or 2009, 74 (11), 4055-4060].

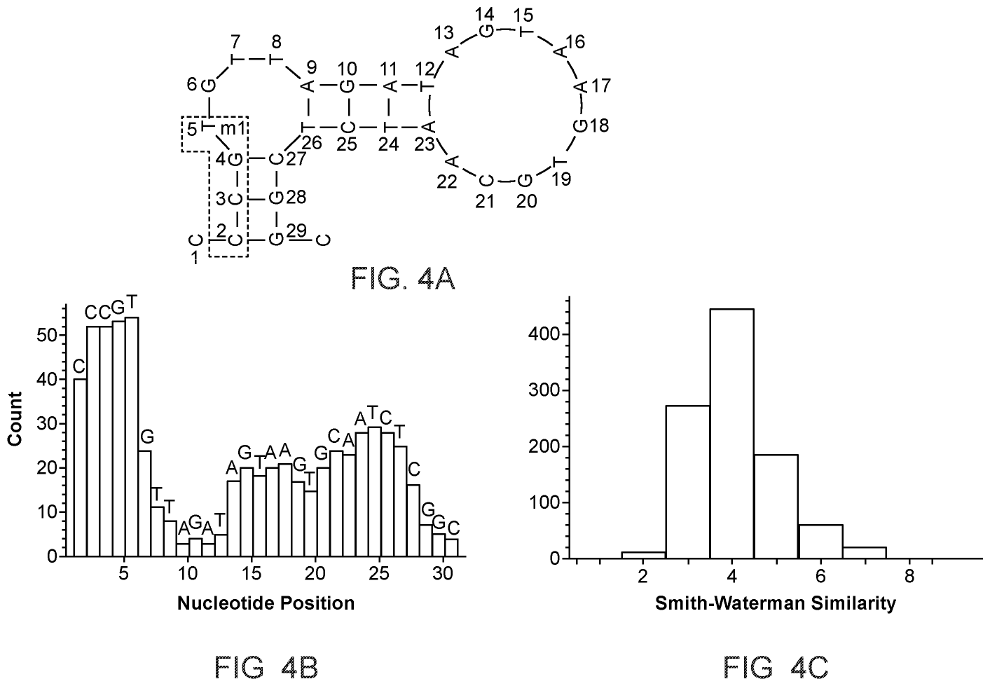

After three rounds of click-PD screening, we were able to identify a non-natural aptamer sequence, NNG (Table S3), which exhibited a K_{D} of 1.9 mM—approximately an order of magnitude better than the best natural DNA aptamer to glucose [Nakatsuka et al., Science 2018, 6750 (September), 1-11] (Figure S12). We next sought to identify the minimal binding region of NNG, as the full-length sequence contains twenty modified bases and would complicate solid phase synthesis of the aptamer strand for the screen. Additionally, it has been shown that the full-length sequence is often not necessary to retain molecular recognition capabilities [Gao et al., Analytical and Bioanalytical Chemistry 2016, 408 (17), 4567-4573, https://doi.org/10.1007/s00216-016-9556-2]. Secondary structure analysis was used to guide truncation of NNG, and this revealed that the primary structural element was a minimized 30-nucleotide hairpin near the 3′ terminus of the sequence with only eight modified bases, which we hypothesized to be the binding site (FIG. 4A). We generated beads displaying this truncated sequence, NNGmin, and compared its binding performance relative to glu1 using flow cytometry. We observe a 1.7-fold increase in affinity in comparison to the full-length sequence (1.1 mM), confirming that this minimized glu1 min sequence would be suitable for developing a glucose-responsive switch.

FIG. 4: Analysis of the top 1,000 unique glucose SD sequences. A) Predicted secondary structure of the phenylboronic acid-modified glucose aptamer glu1 min. Red bolded Ts denote location of modifications. The boxed region m1 indicates a motif that was highly recurrent among the sequence elements targeted by our top SDs. B) Histogram of the frequency with which each aptamer base-position was complementary to an SD. C) Histogram of Smith-Waterman similarity distances between the glucose aptamer and the reverse-complement of the top 1,000 SD sequences.

Using our ADS screen, we were able to identify non-natural aptamer switches which were responsive to glucose concentrations in the low millimolar range. Although our aptamer is base-modified, we could still utilize the same natural DNA switching strand library, highlighting the generalizability of our strategy. We screened our ADS library against two concentrations of glucose, imaging with four alternating cycles each of buffer, 10 mM glucose, and 100 mM glucose. Interestingly, while we identified a few weakly-performing signal-on switches, glu1 min primarily yielded signal-off switches that exhibited a decrease in fluorescence upon binding glucose. We cannot a priori predict the mechanism by which the switches identified in our screen will function, and the amplitude and direction of the fluorescence response will be dictated by factors such as the nature of the interaction between SD region and aptamer, steric clashing between the switching and aptamer strands, and the three-dimensional structure of the aptamer. Analysis of the filtered data revealed that 200 of the ˜495,000 clusters exhibited a 1.3-fold change in fluorescence intensity upon the addition of glucose, with the most responsive sequence showing a 2-fold change. Although the magnitude of the signal change for the top sequences was modest compared to the ATP screen (approximately 50% lower), we were encouraged to observe similar decreases in fluorescence with both 10 and 100 mM glucose, suggesting that the top switching domains were most dynamic in the low millimolar range.

To gain insight into which bases within the aptamer were key for switching function, we subsequently performed MEME analysis of the top 1,000 SD sequences to identify any conserved motifs. We found that bases 2-5 near the 5′ terminus of glu1min were overwhelmingly targeted by 15.7% of the top sequences (FIG. 4A). A histogram of the frequency with which each base-position in the glucose aptamer was complementary to one of these top-performing SD sequences also showed a prominent peak at the 5′ end, in keeping with the MEME analysis (FIG. 4B). This suggests that the short stem region targeted by this m1 motif is highly amenable to strand-displacement-based switching based on competition with the duplexed SD, where target binding favors the formation of the intramolecular stem region and release of the SD sequence. In contrast to the top ATP switches, the enriched targeting of this motif is consistent with conventional rational-design strategies, wherein the stem that stabilizes the aptamer structure would be preferentially targeted for displacement. This region may also be preferentially targeted because the adjacent loop region contains three tightly-spaced boronic acid modifications in bases 5-8, which we hypothesize act to enhance molecular recognition of glucose by the aptamer. On the other hand, we were surprised to note that the other predicted short stem region between bases 9-12 appears to be the least amenable region of the aptamer for switch design, which would potentially confound heuristic-based rational design approaches. We hypothesize that this difference in impact arises because three of the four base pairs in this predicted stem involve a modified base, which could potentially destabilize this stem and even completely prevent it from forming. Although the boronic acid modification is incorporated at a position facing away from the binding face of the uracil base, the presence of modifications nevertheless results in steric hindrance that has been shown to lower the stability of nucleic acid duplexes [Freier, S. M. and Altmann, K. H., Nucleic Acids Research 1997, 25 (22), 4429-444]. This highlights the unique obstacles in utilizing non-natural nucleic acids for switch development, which we overcome during the screening process as no prior knowledge of the aptamer folding is required.

As with the ATP ADS constructs, sequence alignment analysis revealed that the SD sequences exhibited notably low complementarity with the non-natural glucose aptamer. Approximately 73% of the top 1,000 sequences showed a Smith-Waterman score≤4 to the reverse-complement of the aptamer (FIG. 4C), demonstrating that more than half of the bases within top-performing SD sequences would not be expected to interact with the aptamer through canonical Watson-Crick base-pairing. We hypothesize that the grouping of boronic acid modifications in bases 5-8 and 9-12 likely contributes to the low SD complementarity due to the destabilizing effect of the base modification, which results in alternative interactions which cannot be predicted or modeled with current tools. Thus, our analysis again shows that many of the best-performing SD sequences are largely composed of bases that participate in non-canonical binding modes that would most likely not be identified in a rational design-based effort.

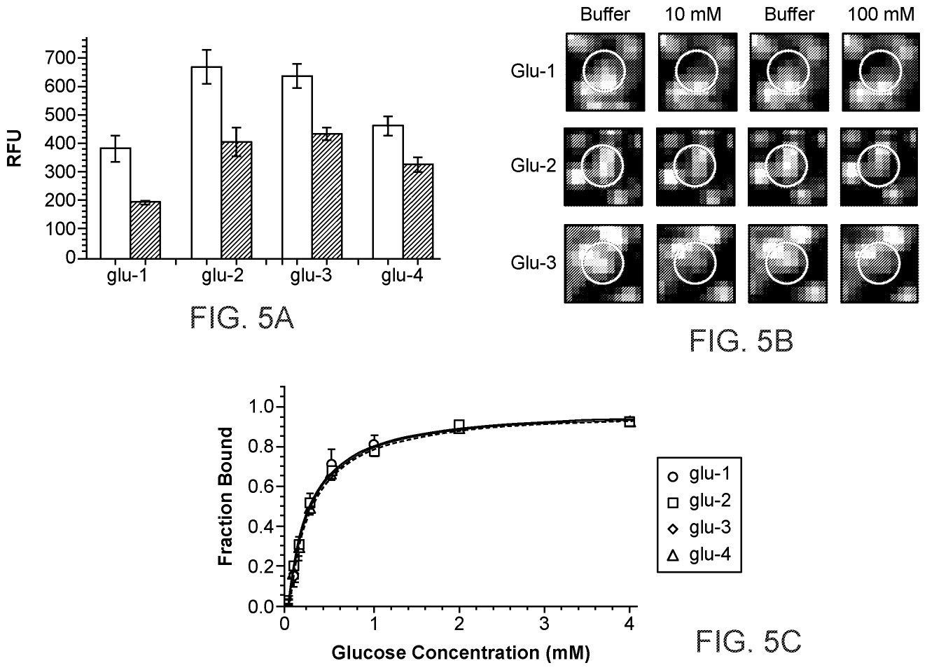

Finally, we identified and experimentally validated four glu1min-based switch constructs with low millimolar affinity for glucose. We chose four switches (Glu ADS1-4; Table S4) that exhibited the greatest signal-off response (1.3-2-fold decrease) in the presence of target, which we validated via cluster image analysis (FIG. 5A, B). We then assessed the glucose affinity of these four sequences in a plate reader assay, as described for the ATP screen above. All four sequences displayed the expected dose-dependent decrease in fluorescence, with a maximum signal change of approximately two-fold-comparable to that observed in the flow-cell. All four ADS constructs possessed affinities on the order of ˜0.3 mM, making these constructs ˜30-fold more sensitive to glucose than a previously published natural DNA-based switch (FIG. 5C). We were surprised to observe that our ADS constructs possessed affinity that was comparable and even slightly superior to that of both the original glu1 aptamer and the glu1min derivative. Traditionally, the conversion of an aptamer to a strand displacement-based switch results in an increase to the apparent K_{D}, as higher target concentrations are needed to outcompete the complementary strand. These results highlight that the non-canonical interactions identified using our screening method could prove advantageous in the identification of ADS switch constructs which retain the affinity of their parent aptamer, and show that base-modified DNA can be advantageous for the development of high-affinity switches for challenging small-molecule targets.

FIG. 5. Identification and characterization of phenylboronic acid-modified aptamer switches for glucose. A) Analysis of the top four aptamer switch clusters identified in our flow-cell screen. Orange and blue bars represent cluster intensity in buffer and 10 mM glucose, respectively. Error bars represent the standard deviation of the measurements (n=4). B) Extracted images of clusters glu-1, -2, and -3 (red circles) on the MiSeq flow cell for both the buffer and glucose cycles. C) Validation of the glucose affinity of the four aptamers shown in panel A. 50 nM labeled ADS construct was incubated with various concentrations of glucose and the fluorescence signal was measured on a plate reader. The solid lines represent the fitted single binding site model. Error bars represent the standard deviation of three measurements.

### CONCLUSION

In this work, we describe a massively parallel screening strategy for generating target-responsive molecular switches from an existing aptamer in a single experiment. Importantly, our strategy does not require any prior knowledge of aptamer structure and eliminates the need for computer modeling or design heuristics. To demonstrate the generalizability of our method, we converted two aptamers—a natural DNA aptamer for ATP, and a newly-selected phenylboronic acid-modified DNA aptamer for glucose—into molecular switches. Many of the resultant aptamer switches were either predicted not to fold or yielded inconsistent results when analyzed with various secondary-structure prediction software tools, which indicates that these switch constructs would have been overlooked by rational design-based approaches. Indeed, several of the aptamer sequence elements preferentially enriched for in our screen would have been counter-intuitive based on conventional design heuristics—for example, targeting one of the loop structures in the ATP aptamer, or favoring one predicted stem versus another in the glucose aptamer. We have also demonstrated that our screen is applicable to base-modified aptamers for which in silico prediction of aptamer structure may not be feasible. Since our approach is predicated on the use of SD libraries composed entirely of natural DNA, this same system should be applicable to RNA aptamers or other non-natural aptamer chemistries such as xeno-nucleic acids (XNA) [Rangel et al., In Vitro Selection of an XNA Aptamer Capable of Small-Molecule Recognition. 2018, 46 (16), 8057-8068], as long as the aptamer molecule is capable of stable hybridization with a DNA-based switching strand. Even as the number of published aptamers steadily grows, it has remained a challenging and time-consuming task to convert those aptamers into functional molecular switches. This work addresses this fundamental challenge by relying on unbiased high-throughput screening to cover the full sequence space available for a DS construct, rather than relying on rational design informed by potentially faulty assumptions of aptamer structure and function. Our approach is generalizable and should therefore help accelerate the generation of functional aptamer-based switches, thereby facilitating the creation of novel biosensors for use in a broad range of applications.

### Materials

Terminal transferase (TdT) was ordered from New England Biolabs (#M0315L). DdeI restriction enzyme was ordered from New England Biolabs (#R0175L). Cy3-labeled ddUTP (5-propargylamino-ddUTP-Cy3) was ordered from Jena Bioscience (#NU-1619-CY3) and unlabeled ddTTP (2′,3′-dideoxythymidine-5′-triphosphate) was ordered from TriLink Biotechnologies (#N-4004). ATP (adenosine 5′-triphosphate) was ordered from Thermo Fisher Scientific (#R0441) and glucose was purchased from Sigma-Aldrich (#G8270). The strands for the switch screen were all purchased HPLC-purified from Integrated DNA Technologies (IDT); all purchased sequences are presented in Tables S1 and S3. The switching domains that were validated via plate-reader were purchased from the Stanford Protein and Nucleic Acid facility and the sequences are presented in Table S2. Streptavidin beads for the proof-of-concept TdT and DdeI experiments were purchased from Thermo Fisher Scientific (#88816).

### Methods

### Conjugation of Glucose to Magnetic Beads:

All reagents were purchased from Sigma Aldrich unless otherwise noted. Glucose was immobilized onto alkyne-coated magnetic beads (Jena Bioscience) using CuAAC click chemistry. 400 μL of beads were washed three times with 400 μL of 0.1% Tween-20 in PBS. The washed beads were resuspended in 1×PBS, 30 μL of a pre-prepared mixture of 0.1 M CuSO4/0.2 M Tris (3-hydroxypropyltriazolylmethyl)amine (THPTA), and 30 mM azido-PEG4-β-D-glucose (BroadPharm), and H₂O to a final volume of 225 μL. 25 μL of freshly-prepared 50 mM sodium ascorbate was added to the reaction mixture for a final concentration of 5 mM. The solution was degassed using N₂ for 5 minutes, then reacted for 1 hr at room temperature with rotation. Beads were then washed three times and resuspended with 400 μL 1× SELEX buffer (100 mM NaCl, 2 mM MgCl₂, 5 mM KCl, 1 mM CaCl₂), 0.02% Tween 20, 20 mM Tris-HCl, pH 7.5).

### Conjugation of AlexaFluor 647 to Magnetic Beads:

AlexaFluor 647 was immobilized onto Dynabeads M-270 amine magnetic beads (ThernoFisher) using standard amine reactive chemistry. 400 μL of amine beads were washed three times with 400 μL 0.1% Tween-20 in PBS. 1 mg Alexa Fluor 647 NHS ester (ThermoFisher) was resuspended in DMF to a final concentration of 10 μg/μL. The washed beads were resuspended in a 400 μL solution comprising 24 μL Alexa Fluor 647 NHS-ester stock in 1×PBS. The bead mixture was incubated for 2 hrs at room temperature (RT) with rotation. Beads were then washed three times and resuspended with 400 μL 1× SELEX buffer.

### Pre-Enrichment by SELEX for Glucose:

Two rounds of positive selection were performed with bead-immobilized glucose. All bead washing steps were performed using a Dynamag-2 magnetic separation stand (ThermoFisher). 1 nmol of the GluPD library (Table S3) was folded in 200 μL of 1× SELEX buffer by heating to 95° C. for 5 min, cooling at 4° C. for 10 min, and then incubating at 25° C. for 10 min. In the first round of pre-enrichment, we included a counterselection step where the folded library was incubated with 5 nmol (20 μl) of Alexa Fluor 647-conjugated magnetic beads for 1 hr at RT with rotation to eliminate sequences that preferentially bind the fluorophore. These beads were then washed twice with 100 μl 1× glucose selection buffer, and the total 200 μl of buffer that was washed from the Alexa Fluor 647 beads were then incubated with 5 nmol (20 μl) of glucose-conjugated magnetic beads which were pre-washed as described above for the AlexaFluor 647 beads for 1 hr at RT with rotation. Beads were washed twice with 100 μl 1× SELEX buffer and eluted into 100 μl water by heating to 95° C. for 5 min. Recovered DNA was purified with a Qiagen MiniElute cleanup kit and eluted in 10 μl water. We then PCR amplified 1.5 μl of the recovered library with 10 μl 2× GoTaq PCR mix, 200 nM Glu FP, 200 nM biotinylated Glu RP, and H₂O up to a final reaction volume of 50 μl using the following cycling conditions: 95° C. for 3 min, followed by X cycles of 95° C. for 15 s, 53° C. for 30 s, 72° C. for 30 s and finally 72° C. for 2 min. To determine the correct number of cycles for amplification, a pilot PCR was run. 3 μl of the reaction was removed every 3 cycles (15 cycles total), and mixed with 3 μl 2× Novex TBE-urea sample buffer (ThermoFisher) and run on a 10% TBE gel at 200 V for 40 min. The cycle that yielded a product of the correct length without forming undesired products was chosen for the final scaled-up PCR reaction. To generate single-stranded DNA, biotinylated double-stranded DNA was immobilized onto 100 μl MyOne streptavidin C1 magnetic beads according to the manufacturer's protocol in 500 μl 1× Binding and Washing buffer (5 mM Tris-HCl (pH 7.5), 0.5 mM EDTA, 1 M NaCl) in a 1.5 mL tube. Beads were incubated with 100 μl freshly prepared 0.5 M NaOH for 10 minutes at RT. The tube was placed on a DynaMag-2 magnetic rack (Life Technologies) for 2 minutes, and the supernatant was collected. Beads were washed once more with 50 μl 0.1 M NaOH. DNA was recovered from NaOH by adjusting the pH with 25 μl 3M NaOAc, then purified with a Qiagen MiniElute cleanup kit and eluted in 20 μl water.

### Forward Primer (FP) Bead Conjugation Protocol:

500 μL of Dynabeads MyOne Carboxylic Acid magnetic beads (Thermo Fisher Scientific) were washed five times with 500 μL of water on a magnetic rack. The beads were then resuspended in 150 μL of 0.2 mM 5′ amino-PEG Glu FP, 200 mM NaCl, 1 mM imidazole chloride and 250 mM EDC. The mixture was mixed well and sonicated prior to incubation at RT overnight on a rotator. Next, we conjugated PEG12 to the unreacted free carboxyls on the magnetic beads through a two-step NHS/EDC reaction to reduce non-specific interaction with the target proteins. The beads were washed three times with 500 μL of 100 mM MES buffer (pH 4.7). During the last wash step, the beads were incubated for 10 minutes at RT on a rotator. Immediately before use, an 80 mg/mL solution of EDC and a 25 mg/mL solution of NHS were prepared in cold 100 mM MES buffer. The FP beads were then resuspended in equal volumes of NHS and EDC solutions to a final volume of 150 μL. The beads were mixed well and incubated at RT on a rotator for 30 minutes. The beads were washed twice with 500 μL of cold PBS. The activated beads were then resuspended in 150 μl of 20 mM amino-PEG in PBS, mixed well, and incubated for at least 30 minutes at RT on a rotator. The beads were then washed three times for 15 minutes with 500 μL of 1× SELEX buffer in order to quench any amine-reactive NHS esters. Finally, the beads were resuspended in 500 μL of 1× SELEX buffer and stored at 4° C. To verify successful attachment of the primer to the beads, 1 μl of FP beads and 1 μL of 100 μM Alexa Fluor 647-labeled FP complement were mixed in 100 μl SELEX buffer and incubated for 10 minutes at RT on a rotator. The beads were then washed once with 100 μL of 1× SELEX buffer, resuspended in 100 μL 1× SELEX buffer, and run on a benchtop flow cytometer (BD Accuri C6 Plus).

### Emulsion PCR Protocol:

The emulsion PCR process involves the creation of an oil phase and an aqueous phase. The oil phase consists of 4.5% Span-80, 0.4% Tween 80, and 0.05% Triton X-100 in mineral oil (all purchased from Sigma-Aldrich), stored at RT in the dark. The aqueous phase consists of 1×KOD XL buffer, 0.5 U of KOD XL polymerase, 0.2 mM dATP, 0.2 mM dCTP, 0.2 mM dGTP, 0.2 mM aminoallyl dUTP (all purchased from Thermo Fisher Scientific), 10 nM FP, 1 μM RP, 1.5 pM dsDNA enriched glucose library, and ˜3×10⁸ FP-coated magnetic beads (12 μL of FP-bead suspension) in a total volume of 1 mL of water. To create the water-in-oil emulsions, 7 mL of the oil phase was added to a DT-20 tube (IKA) and 1 mL of the aqueous phase was added dropwise over ˜30 seconds while the mixture was stirred at 620 rpm in an Ultra-Turrax device (IKA). The mixture was then stirred on the device for another 5 min. The emulsion was then hand pipetted into ˜80 wells of a 96-well PCR plate (100 μL per well). The plate was then run on an Eppendorf Mastercylcler X50 PCR machine for 40 cycles.

### Emulsion Cleanup:

After PCR, the emulsions were transferred to a 50 mL Falcon tube. 125 μL of 2-butanol (Thermo Fisher Scientific) was added to each well to wash residual emulsion, and the butanol was then transferred to the same 50 mL tube. The tube was vortexed for 30 seconds and then centrifuged at 3000×g for 5 minutes. The supernatant was removed while retaining the pellet of aptamer particles at the bottom of the tube. 1.2 mL of breaking buffer (100 mM NaCl, 1% Triton X-100, 10 mM Tris-HCl, pH 7.5, and 1 mM EDTA) was added to resuspend the particles, and the mixture was transferred to a new 1.5 mL tube. The 1.5 mL tube was vortexed and centrifuged at 21,000×g for 1 minute. Using a magnetic rack, the supernatant was removed with a 1 mL micropipette. Another 1 mL of breaking buffer was added to the particles, which were then transferred to a new tube, and the supernatant was removed as described above for multiple cycles until no residual oil (white film) was visible on the top layer. On the last wash, 150 μL of breaking buffer was added to the sample, which was then transferred to a new 1.5 mL tube. The supernatant was removed, and the aptamer particles were washed three times and resuspended with 500 μL SELEX buffer.

### Boronic-Acid Functionalization of Aptamer Particles:

Aptamer particles recovered from the emulsion PCR were washed twice with 200 μL 1×PBS and resuspended in a 150 μL solution containing 1×PBS, and 30 μL of a 10 mg/mL solution of DBCO NHS ester. The beads were incubated for 2 hrs at RT with rotation. Beads were washed three times and resuspended in 0.1% Tween-20 in PBS. The DBCO-modified aptamer particles were washed three times with 200 μL 0.1% Tween-20 in PBS. The washed beads were resuspended in a 150 μL solution containing 1×PBS and 40 μL of a 1 mg/mL solution of azido-phenylboronic acid. The beads were vortexed and incubated at RT overnight. The boronic acid-modified beads were then washed three times and resuspended with 100 μL 1× SELEX buffer.

### Single-Stranded DNA Generation and Aptamer Particle Quality Control:

The aptamer particles were resuspended in 500 μL of 200 mM NaOH and incubated for 10 min at RT on a rotator. The aptamer particles were washed twice with 500 μL of 100 mM NaOH and then three times with 1 mL of 1× SELEX buffer and finally resuspended in 100 μL of 1× SELEX buffer. To ensure the successful synthesis and monoclonality of the aptamer particles, 1 μL of the aptamer particle solution and 1 μL of 100 μM Alex Fluor 647 Glu RP were incubated in a total volume of 100 μl 1× SELEX buffer for 10 minutes at RT with rotation. The beads were washed once with 100 μl of 1× SELEX buffer, resuspended in 50 μl 1× SELEX buffer, and run on a flow cytometer (BD Accuri C6 Plus).

### Glucose Labeling:

Glucose was conjugated to AlexaFluor 647 or AlexaFluor 488 using CuAAC click chemistry. Solutions were prepared containing 1×PBS, 30 μL of a pre-prepared mixture of 0.1 M CuSO4/0.2 M tris(3-hydroxypropyltriazolylmethyl)amine (THPTA), 30 mM azido-PEG4-β-D-glucose (BroadPharm), 50 μL of a 10 mg/ml stock of either Alexa Fluor 647 alkyne or Alexa Fluor 488 alkyne (Invitrogen), and H₂O to a final volume of 225 μL. 25 μL of a freshly-prepared 50 mM solution of sodium ascorbate was added to the reaction mixture for a final concentration of 5 mM. The solution was degassed using N₂ for 5 minutes, then reacted for 1 hr at room temperature with rotation. After the labeling reaction was complete, the samples were dialyzed in water using Tube-O-DIALYZER Micro 1K MWCO (G-Biosciences).

### Click-PD Screening for Glucose-Specific Aptamers

Three rounds of particle display were performed with Alexa Fluor 647-labeled glucose, and one final round was performed with Alexa Fluor 488-labeled glucose to eliminate any sequences that may have evolved to preferentially bind the fluorophore. Prior to sorting, a flow cytometry binding assay was performed with multiple concentrations of glucose (250, 500, 750, 1,000 μM) to determine which resulted in sufficient binding to the target. For the assay, 1 μL of the boronic acid-modified aptamer particle solution was added to a solution containing the labeled glucose at the specified final concentrations in a total reaction volume of 50 μL of 1× SELEX buffer and incubated in the dark for 1 hr at RT on a rotator. Samples were then washed with 100 μL 1× SELEX buffer and resuspended in cold 1× SELEX buffer for analysis. The optimal concentration of glucose for sorting was determined as the highest concentration that yielded >1% binding to labeled glucose. For sorting, aptamer particles were folded in 1 mL SELEX buffer by heating to 95° C. for 5 min and leaving to cool to room temperature for 30 min. and then incubated with 750 μM labeled glucose in the dark for 1 h at room temperature with rotation. The beads were washed twice and resuspended in 1 mL cold 1× SELEX buffer and then analyzed using a BD FACS Aria III. The sort gate was set to collect 0.5% (round 1) or 0.3% (rounds 2 and 3) of aptamer particles that showed high affinity for glucose by identifying those particles with the greatest shift in the APC channel (rounds 1 and 2) or FITC channel (round 3). After sorting, the collected aptamer particles were resuspended in 20 μL PBS and the aptamers were amplified by PCR using the conditions described above.

### High-Throughput Sequencing Protocol:

For each aptamer pool sequenced, adaptor primers were first added. 10 ng of double-stranded DNA was subjected to eight cycles of PCR using the same conditions described above. A 2× GoTaq Master Mix was used (Promega, M7132) with 1 μM of each primer in a total reaction volume of 100 μL. The sequencing primers were added by using a Nextera XT kit (Illumina) and following the provided instructions. Samples were quantified using a Qubit fluorometer and sent to the Stanford Functional Genomics Facility for sequencing on an Illumina MiSeq.

### Synthesis of Aptamer Particles for Affinity Measurements:

Aptamers were coated onto beads by preparing a 100 μL PCR reaction consisting of 10 μL 10×KOD XL buffer, 2 μL dNTP mix of 10 mM dATP, dGTP, dCTP, aminoallyl dUTP each, 1 μL of 10 UM Glu FP, 10 μL of 10 μM Glu RP, 2 μL of 100 pM aptamer template, 2 μL of KOD XL polymerase, and water to the final volume. 30 PCR cycles were conducted using the conditions described above, and the beads were washed and converted to single-stranded DNA as described above for the emulsion PCR protocol. Beads were washed and resuspended in 50 μL of SELEX buffer prior to storage at 4° C. A 50 μL binding reaction was prepared with 1 μL of the aptamer particle solution and the required volume of the Alexa Fluor 647-labeled glucose stock in 1× SELEX buffer. The samples were incubated on a rotator at RT for 1 hr. The beads were washed twice with 100 μL cold SELEX buffer and resuspended in 50 μL SELEX buffer. The sample was gently mixed via pipette, sonicated briefly, and then immediately run on a flow cytometer (BD Accuri C6 Plus).

### Preparation of the Switching Strand Library:

Prior to running the high-throughput switch screen, the switching strand library must be prepared for MiSeq sequencing. This process only needs to be done once, as the prepared library should be sufficient for many (˜50) runs and is not dependent on the aptamer being used. To prepare the library for sequencing, a Nextera XT DNA Library Preparation Kit was used (Illumina, #FC-131-1024) and the kit instructions were followed. Kit indices N703 and S517 were used, and the final double-stranded PCR product was checked via native PAGE and quantified using a Qubit fluorimeter prior to sequencing.

### Validation of TdT and DdeI Enzymes on Beads:

Prior to MiSeq screening, the TdT labeling reaction and enzymatic cleavage reaction using DdeI were validated using streptavidin-functionalized magnetic beads and a bench-top flow cytometer (BD Accuri C6 Plus Flow Cytometer).

To validate the DdeI restriction enzyme, we utilized a 5′-biotinylated test strand (see Table S1) that was 3′-labeled with Cy3. The test strand contained the same poly-T linker, N10 SD region, and reverse-primer complement region (which contains the DdeI cut site) as the switching strand library that was utilized in the screen. The test strand was captured onto streptavidin beads and then we then annealed the reverse-primer at 100 nM, washed the beads, and incubated the beads with the DdeI enzyme mixture (3 μL DdeI enzyme, 10 μL 10× Cutsmart buffer, 87 μL water) for ten minutes at 37° C. By comparing the fluorescence of the beads before and after the addition of the restriction enzyme, we were able to confirm that DdeI efficiently cleaved the reverse-primer complement region from the aptamer.

To validate the TdT labeling step, we checked the incorporation of a Cy5-labeled ddUTP into the 20-nucleotide biotinylated reverse-primer sequence. The primer sequence was subjected to a test TdT reaction in solution (5 μL 10×TT buffer, 1 μl 10 UM biotin RP, 5 μL CoCl₂, 15 μL 1 mM Cy5 ddUTP, 5 μL TdT enzyme, 29 μL water (reagents from New England Biolabs)) and incubated at 37° C. for 0, 30, 60, or 90 minutes. Samples from these various time-points were then captured on streptavidin magnetic beads to analyze reaction efficiency, which could be assessed by a shift in the red-channel fluorescence corresponding to successful incorporation of Cy5-ddUTP. Analysis of these samples by flow cytometry showed that the reaction proceeded to completion quickly, with a large shift in fluorescence observed after 30 minutes and no significant changes after longer incubation times.

### High-Throughput ADS Screening:

The high-throughput ADS screen was conducted using the custom-built N2A2 system we described previously [Wu et al., Automated Platform for High-Throughput Screening of Base-Modified Aptamers for Affinity and Specificity. 2020, 1-23]. The DdeI enzyme solution (10 μL DdeI enzyme stock, 30 μL 10× Cutsmart buffer, 260 μL water), complement strand solution (3 μL of 100 μM switch complement strand, 40 μL 10× Cutsmart buffer, 356 μL water), blocking TdT solution (30 μL TT buffer, 40 μL CoCl₂ buffer, 45 μL 2 mM dTTP, 16 μL TdT enzyme, 259 μL water), and Cy3-TdT labeling solution (30 μL TT buffer, 40 μL CoCl₂ buffer, 45 μL 1 mM Cy3-ddUTP, 16 μL TdT enzyme, 259 μL water) were all added into empty locations on the MiSeq reagent cartridge. Water, 50 mM NaOH with 1% SDS, selection buffer (20 mM Tris-HCl, 120 mM NaCl, 5 mM KCl, 1 mM MgCl₂, 1 mM CaCl₂), and 0.01% Tween-20 in nuclease-free water), FM buffer (100 nM FM comp 532 and 100 nM FM comp 660 in selection buffer), aptamer solution (100 nM aptamer in FM buffer), and target solutions (ATP or glucose in selection buffer) were all hooked up to the external multiport valve (Valvo Instruments).

The MiSeq XML files and folder agent (see attached files) were altered to conduct three different types of cycles: a switch construction cycles, a buffer cycle, and a target addition cycle. Unless otherwise mentioned, all steps were conducted at 22° C. In the switch construction cycle, the flow-cell is first blocked with ddUTP by flowing in the TdT blocking solution for 45 minutes at 37° C. This is repeated once more. The flow-cell is then washed with the NaOH solution, and then selection buffer. The complement strand solution is then injected onto the flow-cell and allowed to incubate for 15 minutes. Next, the DdeI solution is injected and allowed to incubate for 30 minutes at 37° C. These two steps (complement strand and DdeI incubation) are repeated once again. The flow-cell is once again washed with NaOH solution and selection buffer prior to the final Cy3 labeling of the switching strand library. In this step, the labeling TdT solution is added to the flow-cell for 45 minutes at 37° C. The step is repeated once more, and the switch construction cycle is completed after washing the flow-cell with NaOH solution and then selection buffer.

The buffer cycle involves annealing the quencher-labeled aptamer onto the switching library clusters and then imaging the flow-cell in buffer. The first step of the buffer cycle is to anneal the quencher-labeled aptamer. The aptamer solution is injected onto the flow-cell and then the flow-cell undergoes a temperature anneal (80° C. for 7.5 minutes, 70° C. for 2.5 minutes, 60° C. for 2.5 minutes, 50° C. for 2.5 minutes, 40° C. for 2.5 minutes, 30° C. for 2.5 minutes, 22° C. for 15 minutes). The flow cell is then washed with selection buffer and the clusters are imaged to determine the switch cluster intensities in the present of buffer without target.

In the target addition cycle, the target solution is injected onto the flow cell and then incubated for 5 minutes. This is repeated twice more for a total incubation time of 15 minutes between the target solution and the switch clusters on the surface of the flow cell. The flow-cell is then imaged to determine the switch cluster intensities in the present of target. Prior to beginning the next buffer cycle, the flow-cell is washed with the NaOH solution and then selection buffer to remove the target and aptamer strands from the flow-cell.

### Evaluation of Switches Via Plate-Reader:

For the plate-reader assay of the ATP and glucose aptamer switch constructs, we first annealed the Cy3-labeled switching strands to the quencher-labeled aptamer. The two strands were mixed together at a 1:1 ratio at a final concentration of 50 nM in 1× selection buffer. The solution was heated to 95° C. for 5 minutes and then cooled at RT for 20 minutes. For glucose switches, strands were annealed using a slow cooling from 95-25° C. by decreasing the temperature 2° C. every 30 s. 200 μL of the annealed switch constructs were then incubated for 10 minutes at RT with various concentrations of ATP or glucose in a black 96-well plate (Sigma-Aldritch, #CLS3925). The measurements were taken at 25° C. on a Synergy H1 microplate reader (BioTeK) using a Cy3 filter cube (545 nm excitation, 575 nm emission). The experiment was conducted in quadruplicate, where each well corresponded to an independent incubation of the aptamer switch with target.

### Curve Fitting and Normalization:

In order to normalize the binding curves and to determine the observed dissociation constants for the molecular switches, Wolfram Mathematica (Version 12.0.0.0) was used to fit binding models to the plate-reader data using the NonlinearModelFit function. Since the ATP aptamer has two binding sites, a two-site independent binding model was used (Equation 1) and adapted for the fluorescence measurements taken on the plate-reader (Equation 2). In the first equation, K₁ is the dissociation constant of the first ATP target binding, while K₂ represents the dissociation constant of the second ATP target binding to the aptamer switch, and fᵣ is the fraction bound. In the second equation, y is the relative fluorescence signal detected on the plate-reader, Bₘₐₓ is the maximum signal at 100% binding, and y₀ is the initial fluorescence of the aptamer switch construct in the absence of target.

$${f}_{r} = \frac{{K}_{1} [ ATP ] + {K}_{1} {{K}_{2} [ ATP ]}^{2}}{1 + {K}_{1} [ ATP ] + {K}_{1} {{K}_{2} [ ATP ]}^{2}} \qquad Equation 1$$

$$y = ( {B}_{max} - {y}_{0} ) \frac{{K}_{1} [ ATP ] + {K}_{1} {{K}_{2} [ ATP ]}^{2}}{1 + {K}_{1} [ ATP ] + {K}_{1} {{K}_{2} [ ATP ]}^{2}} + {y}_{0} \qquad Equation 2$$

For the glucose aptamer, a single-site binding model was utilized (Equation 3) and adapted for the fluorescence measurements taken on the plate-reader (Equation 4).

$${f}_{r} = \frac{[ Glucose ]}{[ Glucose ] + {K}_{D}^{eff}} \qquad Equation 3$$

$$y = ( {B}_{max} - {y}_{0} ) \frac{[ Glucose ]}{[ Glucose ] + {K}_{D}^{eff}} + {y}_{0} \qquad Equation 4$$

### SI Tables:

TABLE S1
Sequences used in the switch screen.
Z represents a boronic acid-modified U base.
Q represents the Iowa Black FQ quencher.

| Name | Sequence |
|---|---|
| ADS library | TCGTCGGCAGCGTCAGATGTGTATAAGAGACAG-T(25)-N(10)-TCTCAG |
| | CTGTCTCTTATACACATCTCCGAGCCCACGAGAC (SEQ ID NO: 1) |
| ADS | GTCTCGTGGGCTCGGAGATGTGTATAAGAGACAGCTGAGA (SEQ ID NO: 2) |
| complement | |
| strand | |
| ATP aptamer | QCACCTGGGGGAGTATTGCGGAGGAAGGTTTCTGTCTCTTATACACATCT |
| | GACGCTGCCGACGA (SEQ ID NO: 3) |
| Glucose | QCCCGZGZZAGAZAGZAAGZGCAAZCZCGGCTTTCTGTCTCTTATACACAT |
| aptamer | CTGACGCTGCCGACGA (SEQ ID NO: 4) |
| Ddel Test | Biotin-T(20)-N(10)- |
| strand | TCTCAGCTGTCTCTTATACACATCTCCGAGCCCACGAGAC-Cy5 (SEQ ID |
| | NO: 5) |
| FM | TCGTCGGCAGCGTCAGATGTGTATAAGAGACAGAACCAGGAGCAGGCCAAAAAG |
| | CGCGCAAGAATTCCTGTCTCTTATACACATCTCCGAGCCCACGAGAC (SEQ ID |
| | NO: 6) |
| FM 532 | /5Alex532N/TTGCGCGCTTTTTGGCCTGCTCCTGGTT (SEQ ID NO: 7) |
| comp | |
| FM 660 | /5Alex660N/TTGCGCGCTTTTTGGCCTGCTCCTGGTT (SEQ ID NO: 8) |
| comp | |

TABLE S2
ATP switching strands

| Name | Sequence |
|---|---|
| atp-1 | TCGTCGGCAGCGTCAGATGTGTATAAGAGACAGTTTTTTTTTTTTTTTTTTT |
| | TTTTTTCGACGCGTAATC-[Cy3] (SEQ ID NO: 9) |
| atp-2 | TCGTCGGCAGCGTCAGATGTGTATAAGAGACAGTTTTTTTTTTTTTTTTTTT |
| | TTTTTTACTCCAGCGATC-[Cy3] (SEQ ID NO: 10) |
| atp-3 | TCGTCGGCAGCGTCAGATGTGTATAAGAGACAGTTTTTTTTTTTTTTTTTTT |
| | TTTTTTCGTAGCTCACTC-[Cy3] (SEQ ID NO: 11) |
| atp-4 | TCGTCGGCAGCGTCAGATGTGTATAAGAGACAGTTTTTTTTTTTTTTTTTTT |
| | TTTTTTCGCATCCAGGTC-[Cy3] (SEQ ID NO: 12) |
| atp-5 | TCGTCGGCAGCGTCAGATGTGTATAAGAGACAGTTTTTTTTTTTTTTTTTTT |
| | TTTTTTGAGCAATATATC-[Cy3] (SEQ ID NO: 13) |
| atp-6 | TCGTCGGCAGCGTCAGATGTGTATAAGAGACAGTTTTTTTTTTTTTTTTTTT |
| | TTTTTTATGCGCTACTC-[Cy3] (SEQ ID NO: 14) |
| atp-7 | TCGTCGGCAGCGTCAGATGTGTATAAGAGACAGTTTTTTTTTTTTTTTTTTT |
| | TTTTTTCCGGGGGCTATC-[Cy3] (SEQ ID NO: 15) |
| atp-8 | TCGTCGGCAGCGTCAGATGTGTATAAGAGACAGTTTTTTTTTTTTTTTTTTT |
| | TTTTTTCCGGGGGCGATC-[Cy3] (SEQ ID NO: 16) |

TABLE S3
Sequences used in glucose aptamer selection

| Name | Sequence |
|---|---|
| GluPD | GCGCATACCAGCTTATTCAATTNNNNNNNNNNNNNNNNNNNNNNNN |
| library | NNNNNNNNNNNNNNNNNNNNNNNNNNAGATAGTAAGTGCAATCTC |
| | GGC (SEQ ID NO: 17) |
| Glu FP | GCGCATACCAGCTTATTCAATT (SEQ ID NO: 18) |
| Glu RP | GCCGAGATTGCACTTACTATCT (SEQ ID NO: 19) |
| Amino-PEG | /5AmMC6//iSp18/GCGCATACCAGCTTATTCAATT |
| Glu FP | (SEQ ID NO: 20) |
| AF647-Glu | /5Alex647N/AATTGAATAAGCTGGTATGCGC |
| RP | (SEQ ID NO: 21) |
| Bio-Glu RP | /5Biosg/GCCGAGATTGCACTTACTATCT (SEQ ID NO: 22) |
| AF647 Glu | /5Alex647N/AATTGAATAAGCTGGTATGCGC |
| FPC | (SEQ ID NO: 23) |
| NNG | GCGCATTACCAGCTTATTCAATTGTATCCTTAGTCCCAATGTCTCC |
| | TGGCAACTCGCGCAGTGCCCCGTGTTAGATAGTAAGTGCAATCTCG |
| | GC (SEQ ID NO: 24) |
| NNG min | CCCGTGTTAGATAGTAAGTGCAATCTCGGC (SEQ ID NO: 25) |

TABLE S4
Glucose switching strands

| Name | Sequence |
|---|---|
| glu-1 | TCGTCGGCAGCGTCAGATGTGTATAA |
| | GAGACAGTTTTTTTTTTTTTTTTTTT |
| | TTTTTTTAGGGCAGGCTC-[Cy3] (SEQ ID NO: 26) |
| glu-2 | TCGTCGGCAGCGTCAGATGTGTATAA |
| | GAGACAGTTTTTTTTTTTTTTTTTTT |
| | TTTTTTCAGGACAGGCTC-[Cy3] (SEQ ID NO: 27) |
| glu-3 | TCGTCGGCAGCGTCAGATGTGTATAA |
| | GAGACAGTTTTTTTTTTTTTTTTTTT |
| | TTTTTTTACCAGGAGCTC-[Cy3] (SEQ ID NO: 28) |
| glu-4 | TCGTCGGCAGCGTCAGATGTGTATAA |
| | GAGACAGTTTTTTTTTTTTTTTTTTT |
| | TTTTTTACCAGGAGCTTC-[Cy3] (SEQ ID NO: 29) |

### Example 2

Herein we describe a novel in vitro evolution method coupled with high-throughput screening technology to isolate rationally designed aptamer switches de novo that are directly amenable for real-time measurement. We utilize a double restriction enzyme approach, in which we leverage separate cleavage reactions to eliminate and partition non-functional sequences. We model our library according to our previously described ISD architecture (Wilson, B. D., Hariri, A. A., Thompson, I. A. P. et al. Independent control of the thermodynamic and kinetic properties of aptamer switches. Nat Commun 10, 5079 (2019)) so that the resultant aptamers can be tuned according to previously described design heuristics and the unimolecular design makes is ideal for real-time applications. This method is advantageous as it overcomes laborious trial and error efforts associated post-selection engineering techniques and is not limited by the narrow scope of existing aptamer targets. Further, the direct selection of a rationally designed architecture such as ISD makes further kinetic and thermodynamic optimization facile for any desired downstream application. We then utilize our aptamer array technology to screen our enriched ISD libraries for switching capability in a massively parallel manner. We obtain ISD switches for two challenging small-molecule targets, glucose and 3HB to demonstrate the generalizability of our method. We show that we can obtain linked aptamer switches with affinities in the physiologically-relevant range in six rounds of selection, approximately half as many rounds required by traditional selection methods requiring solid supports.

### Results and Discussion

We modeled our library design after our previously reported ISD strategy using a structured hairpin containing a randomized “loop” region and displacement stem region. The loop region of the library is comprised on the random region and a polyT linker, which is flanked by two primer binding sites which hybridize to form the stem of the hairpin (FIG. 6). Both of the primer binding sites contain half of the recognition sequence for BamHI so when the hairpin is hybridized, the double stranded cut site can be cleaved by the enzyme. The 3′ end of the library also contains part of the recognition sequence for the DdeI enzyme, which can be utilized in a pre-selection step to selectively cleave sequences which cannot form the ISD structure to begin with. During the selection process, the library is hybridized so that it begins in the hairpin configuration, and after target is introduced, active ISD switches will undergo a conformational change in which both sides of the stem become spatially separated. Conversely, inactive sequences will remain in the original hairpin, leaving the double stranded BamHI recognition sequence intact, removing the majority of both primer binding sites once cleaved. The resulting pool is subjected to amplification, where only the active ISD switches, because they still have the primer binding sites, will be selectively amplified for the next round of selection. Additionally, enzyme-based partitioning is label free which enabled us isolate switches without having to modify the target of interest. Further, because restriction enzymes are highly specific and efficient, the use of two different enzymes to partition non-functional sequences should greatly reduce background and increase round to round enrichment compared to conventional SELEX methods.

For our initial ISD library design we chose a random region length of 30 nt as we hypothesized this would allow for sufficient diversity while also avoiding long sequence lengths which might complicate downstream synthesis. The primer binding sites were designed to be 18 nt in length while only 12 of those hybridize to form the stem of hairpin. The remaining 6 nt were designed to be mismatches, so that while they would not contribute to the stem there would be no need for additional steps to reintroduce the remaining bases needed for PCR amplification. A polyT length of 5 was chosen to provide enough linker that could be subsequently optimized, but not adding so many bases that the increase in loop size would destabilize the hairpin according to ISD design principles. We began by determining the cleavage efficiency of this library which we quantify as the percentage of cleaved and uncleaved sequences. This library (35-12) named for the loop and displacement lengths respectively, was cleaved by BamHI to evaluate the reaction efficiency (79%). We hypothesize that the 21% uncleaved is due to non-hairpin forming sequences which are not capable of forming the stable duplexed stem region. During the selection process we monitor our active sequences as the percent uncleaved, so any of sequences that do not form the hairpin in the naïve library pool have the potential to convolute the enrichment and lead to false positives. To overcome this, we added part of a recognition sequence to the 3′ end of the library for the DdeI restriction enzyme. This allows us to introduce a 10-mer strand which is complementary to the 3′ end and once hybridized forms the complete recognition site so that it can be cleaved by DdeI. Those sequences that begin in an open state will hybridize to the 10-mer complement while the hairpins with the 12 nt stem will remain closed as the complement cannot out compete the stem region. This allows for the removal of a 3′ biotin modification, allowing for the selective capture of hairpin forming sequences before the first round of selection. After the inclusion of the DdeI step, we observed a significant decrease in percent uncleaved of the 35-12 library from 21% to 1% which we hypothesized would be better suitable for selection.

Next, we wanted to investigate the performance of BamHI in different various buffer conditions that would result in probes directly transferrable to a real-time measurement application. Since our selection method requires minimal purification steps and is completely in solution, the target incubation and enzyme digestion occur in the same tube and buffer. The manufacturer buffer supplied with the enzyme contains both BSA and 10 mM final concentration of magnesium which are potentially problematic as the presence of BSA during target incubation could inadvertently lead to the development of aptamers to that protein rather than the desired small-molecule and 10 mM Mg2+ is much higher than blood magnesium concentrations. We assayed the cleavage efficiency of our library in buffer conditions with no BSA and various concentrations of magnesium and observed no considerable effect in the omission of BSA. However, we did determine that there was approximately a 0.5% penalty increase in % uncleaved per every 2-fold reduction in magnesium concentration from 10 mM to 1.25 mM. Beginning the selection with background signal, in this case sequences which remain uncleaved due to efficiency of the enzyme in lower magnesium and not because they successfully switch, could be detrimental to the enrichment of functional sequences. Informed by these results, we decided to move forward with a magnesium concentration of 2.5 mM Mg2+ with the added considerations that these conditions would maintain stability of the initial hairpin state as well as encourage proper folding upon target binding. To offset the fact that this magnesium concentration is higher than blood levels, we decided to include a final round during our SELEX process where we drop the concentration of magnesium after the library is thoroughly enriched, so that a slight reduction in the enzyme cleavage efficiency is less likely to result in enrichment of non-functional sequences.

To assess the efficacy of the double enzyme-based ISD SELEX method, we chose glucose as our first target. Glucose remains a target of interest with implications in many diseases, and despite many efforts towards its detection, developing affinity reagents remains a challenge. There have been recent reports of aptamer switches to glucose, however, their sensitivities and optimized buffer conditions are not amenable for the development of real-time probes. We initiated our glucose ISD selection at 20 mM and dropped the concentration to 5 mM over subsequent rounds in order to find switches which are sensitive in the physiologically relevant concentrations of glucose. We analyzed a sample of the BamHI digestion of the library by gel every round to monitor selection progress as an increase in % uncleaved is expected indicating enrichment of active ISD sequences. In the first two rounds we held the concentration at 20 mM and observed an impressive 4-fold increase in % uncleaved (5.1% to 21.1%). In round 3, we lowered the concentration to 5 mM glucose and observed an expected slight decrease in enrichment (12.1%), which was quickly recovered in round 4 (55.3%). We observed a plateau in enrichment by round 5 (62.1%) indicating successful enrichment of the ISD library. At this point, we included one final step as discussed previously to drop the magnesium concentration to 1.25 mM and eliminate any sequences which will not function in the presence of reduced divalent cations.

The enriched library was then prepared for sequencing and assayed for switching behavior using our ISD screen methodology (FIG. 7). The ISD screen, which builds upon our N2A2 and ADS screening technologies (e.g., Wu, D.; et al. Automated Platform for High-Throughput Screening of Base-Modified Aptamers for Affinity and Specificity. bioRxiv 2020, 1-23), enables the direct identification of de novo linked aptamer switches in a massively parallel manner. This process entails three main steps: sequencing, assembly, and characterization. First, clusters of our ISD library were generated on a flowcell surface using Illumina's sequencing by synthesis protocol on a Miseq instrument. Then a short sequence complementary to the 3 ‘end was introduced which allows us to repurpose the DdeI recognition area within the ISD library to remove the excess 5’ nucleotides which were not necessary for formation of the ISD construct. A terminal transferase was then used to introduce fluorescently labeled chain terminating nucleotide to the 5′ terminus of the sequences. In the last step of the assembly, a quencher-labeled strand complementary to the 3′ end of the DNA clusters was introduced while simultaneously folding the library into the ISD hairpin which brought the fluorophore and quencher in proximity and rendered the clusters in a quenched state. Finally, in the characterization step the target was introduced and active ISD switches were identified as the conformational change upon target binding will open the hairpin, causing spatial separation of the fluorophore resulting in a distinct ON signal.

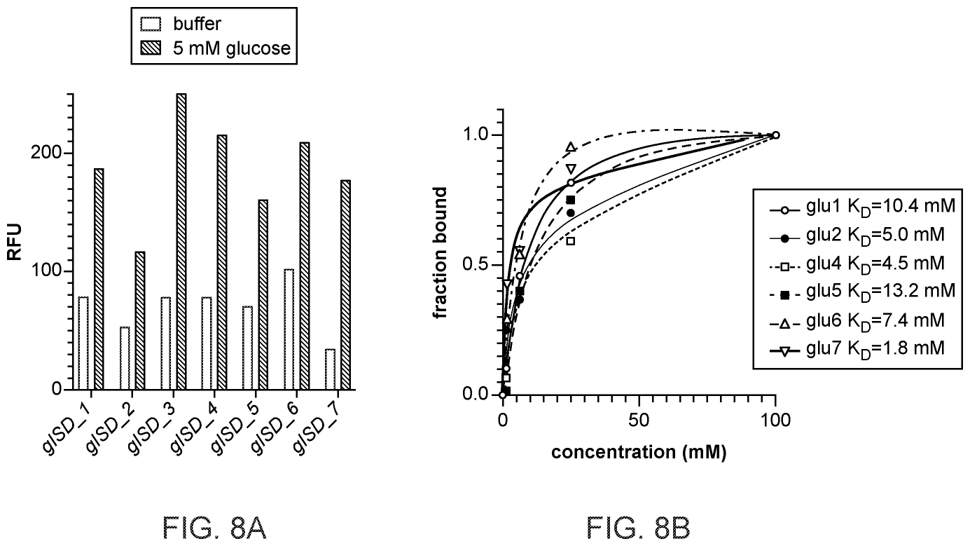

During the screening process, we performed alternating cycles of buffer, 5 mM glucose, and 100 mM glucose to ensure we could identify switches with low mM affinities. We then used the filtered data to identify sequences which exhibited the largest fluorescence increase in the presence of glucose and had replicate clusters to overcome noise within the system. We identified 7 top sequences, all of which displayed significant fluorescence enhancement (2-3×) in response to 5 mM glucose and had greater than 10 replicates (FIG. 8A). We validated the switching capability of these constructs in solution using a plate reader assay. The sequences displayed the expected dose-dependent increase in fluorescence upon the addition of glucose with affinities in the low single digit millimolar range (FIG. 8B).

GluISD_1
(SEQ ID NO: 30)
AGTGGATCCAGCCGGAGGCTGGATTAGAGA
CTGAGGAGCTAGTTTTTGCTGGATCCACT
GluISD_2
(SEQ ID NO: 31)
AGTGGATCCAGCCGGAGGCTGGATTAGAGA
CTGAAGGAGCTAGTTTTTGCTGGATCCACT
GluISD_3
(SEQ ID NO: 32)
AGTGGATCCAGCGGAGCTGGATAAGAGAGA
GGGATACATGCAATTTTTGCTGGATCCACT
GluISD_4
(SEQ ID NO: 33)
AGTGGATCCAGCGGAGCTGGGATAAGAGAG
AGGATACATGCAATTTTTGCTGGATCCACT
GluISD_5
(SEQ ID NO: 34)
AGTGGATCCAGCCGGAGGCTGGATTAAGAG
ACTGAAGAGCTAGTTTTTGCTGGATCCACT
GluISD_6
(SEQ ID NO: 35)
AGTGGATCCAGCCGGAGGCTGGATTAGAGA
CTGAGGGGCTAGTTTTTGCTGGATCCACT
GluISD_7
(SEQ ID NO: 36)
AGTGGATCCAGCGGAGCTGGATAAGAGACA
GGATACATGCAATTTTTGCTGGATCCACT

We observed that the magnitude of signal change between the imager results and plate reader assay were highly correlated, validating that the ISD screening method provides a reliable and accurate method to identify optically active intramolecular switches. Further, we highlight that these switches were validated in low magnesium conditions, eliminating the need for downstream optimization of these switches.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described. All publications, patents, and patent applications cited in this specification are herein incorporated by reference as if each individual publication, patent, or patent application were specifically and individually indicated to be incorporated by reference.

What is claimed is:

1. A method for screening for molecular switches for a target molecule, the method comprising,
(a) providing at least 100 different potential molecular switches comprising a random sequence, each of the at least 100 different potential molecular switches separated from the other on a solid surface or solid support, wherein the potential molecular switches comprise a first nucleic acid linked to a first label and a second nucleic acid linked to a second label, wherein the first label and the second label generate a detectable signal that changes depending on the proximity of the labels to each other;
(b) measuring the detectable signal in the presence and absence of the target molecule, and
(c) identifying molecular switches from the potential molecular switches in which the detectable signal changes depending on the presence of the target molecule, thereby identifying molecular switches for the target molecule.

2. The method of claim 1, wherein the solid surface is a flow cell.

3. The method of claim 2, the providing comprises:
(i) forming the first nucleic acids in a nucleotide sequencing reaction comprising synthesis-by-sequencing, wherein the 5′ end of the first nucleic acids is linked to the flow cell and comprise 5′-3′, a first flow cell primer binding site, the random sequence, an enzyme cleavage site and a second flow cell primer binding site, thereby generating a nucleotide sequencing read for each first nucleic acid,
(ii) for each first nucleic acid, recording the location and nucleotide sequence on the flow cell,
(iii) labelling the first nucleic acids;
(iv) annealing the second nucleic acid to a 5′ portion of the first nucleic acid that does not include the random sequence, wherein the second nucleic acid comprises a sequence that is a reverse complement of the 5′ portion of the first nucleic acid; and
(v) wherein the measuring occurs in the flow cell.

4. The method of claim 3, wherein the 5′ portion comprises the first flow cell primer binding site.

5. The method of claim 3, wherein the first nucleic acid comprises an anchor sequence between the first flow cell primer binding site and the random sequence and wherein the 5′ portion comprises the anchor sequence and the second nucleic acid comprises a reverse complement of the anchor sequence.

6. The method of claim 3, wherein the labelling comprises cleaving the enzyme cleavage site in the first nucleic acids with an enzyme to form a new 3′ end of the first nucleic acids and end labeling the new 3′ end with the first label.

7. The method of claim 6, wherein the end labelling comprises contacting a terminal transferase or ligase to the new 3′ end in the presence of the first label.

8. The method of any one of claims 1-3, wherein first label is a fluorophore and the second label is a quencher.

9. The method of any one of claims 1-3, wherein the first label is a quencher and the second label is a fluorophore.

10. The method of any one of claims 1-3, wherein the first label is a donor fluorophore and the second label is an acceptor fluorophore.

11. The method of any one of claims 1-3, wherein first label is an acceptor fluorophore and the second label is a donor fluorophore.

12. The method of any one of claims 1-3, wherein the plurality of partitions is at least 1000 partitions.

13. The method of any one of claims 1-3, wherein the random sequence is 10-50 (e.g., 20-40, e.g., 25-35) contiguous nucleotides long.

14. The method of claim 5, wherein the anchor sequence is 5-500, e.g., 5-100, 10-50, 12-100, 15-50, or 20-30 contiguous nucleotides long.

15. The method of any one of claims 3-14, wherein the second nucleic acid comprises an aptamer sequence with affinity for the target molecule.

16. The method of claim 15, wherein the aptamer sequence is between the second label and the reverse complement of the anchor sequence

17. The method of claim 15, wherein the switching nucleic acid strand comprises a linker sequence between the random sequence and the reverse complement of the anchor sequence.

18. The method of claim 17, wherein the linker sequence is 1-10 (e.g., 4-6) contiguous nucleotides long.

19. The method of claim 18, wherein the linker sequence is a homopolymer sequence.

20. The method of claim 19, wherein the homopolymer is poly T.

21. The method of any one of claims 15-20, further comprising contacting a first nucleic acid/second nucleic acid combinations identified as a molecular switch in the identifying step to the target molecule and measuring a change detectable signal between the presence and absence of the target molecule.

22. The method of claim 3, wherein the first nucleic acids comprise 5′-3′ the anchor sequence, a stem sequence, the random sequence, a reverse complement of the stem sequence, and the enzyme cleavage site, wherein the stem sequence and the reverse complement of the stem sequence form a double stranded stem in the absence of the target, thereby bringing the first label in proximity to the second label.

23. The method of claim 22, wherein prior to the providing (a), the method comprises enriching for polynucleotides that are molecular switches, wherein the enriching comprises,
(a) providing a plurality of a test nucleic acids having a 3′ and a 5′ end, wherein the test nucleic acid comprises (i) the random sequence and (i) a double stranded stem sequence comprising a double-stranded recognition sequence for a sequence-specific endonuclease and primer binding sequences that include at least part of the double-stranded recognition sequence or is closer to the 3′ and 5′ ends than the double-stranded recognition sequence;
(b) contacting the test nucleic acids with a target molecule and the endonuclease, wherein the endonuclease cleaves the double stranded stem sequence unless the target molecule triggers a conformational shift in the test nucleic acids to cause the stem sequence to disrupt the double stranded stem sequence; and
(c) selectively amplifying intact nucleic acids with primers that anneal to the primer binding sequences, thereby enriching for molecular switches that change conformation in the presence of the target molecule.

24. The method of claim 23, wherein the 3′ end of the test nucleic acids comprises one strand of a second restriction enzyme recognition sequence, and
the enriching further comprises:
(i) contacting the plurality of test nucleic acids with a single-stranded oligonucleotide comprising the reverse complement of the second restriction enzyme recognition sequence, wherein if the double stranded stem sequence is present then the one strand is not available to anneal to the single-stranded oligonucleotide and if the double stranded stem sequence is not present the one strand and the single-stranded oligonucleotide anneal for form the second restriction enzyme recognition sequence; and
(ii) contacting the plurality of test nucleic acids from (i) with the second restriction enzyme, thereby cleaving nucleic acids in which the one strand and the single-stranded oligonucleotide anneal, thereby enriching for nucleic acids that form a stem loop.

25. A method for screening for molecular switches, the method comprising,
(a) providing in a plurality of partitions:
an aptamer nucleic acid comprising:
a first label,
an aptamer sequence with binding specificity for a target molecule,
a first anchor molecule, and
a switching nucleic acid strand comprising:
a second label,
a switch domain sequence,
a second anchor molecule that binds to the first anchor molecule, and
a linker sequence between the switch domain sequence and the anchor sequence; and
wherein the first label and the second label generate a detectable signal that changes depending on the proximity of the labels to each other,
wherein the switch domain sequence of the switching nucleic acid strand is different between partitions, such that at least a majority of partitions contain unique switch domain sequences;
(b) binding the first and second anchor molecules in the partitions;
(c) measuring the detectable signal in the partitions in the presence and absence of the target molecule; and
(d) identifying partitions in which the detectable signal changes depending on the presence of the target molecule, thereby identifying partitions containing a switch domain/aptamer sequence combination that functions as a molecular switch for the target molecule.

26. The method of claim 25, wherein the first anchor molecule is an anchor sequence and the second anchor molecule is a reverse complement of the anchor sequence.

27. The method of claim 25,
wherein (a) comprises providing in the plurality of partitions, the switching nucleic acid strand, wherein the switch domain sequence of the switching nucleic acid strand is different between partitions; and
the method further comprises nucleotide sequencing the switching nucleic acid strands in the partitions and recording the location the sequences to their respective partitions;
providing the aptamer nucleic acids in the partitions; and then
performing the hybridizing, the measuring and the identifying.

28. The method of claim 25, wherein the switching nucleic acid strand has a 3′ end and the first label is linked to the 3′ end and the aptamer nucleic acid has a 5′ end and the second label is linked to the 5′ end.

29. The method of claim 25, wherein the anchor sequence is 5-500, e.g., 5-100, 10-50, 12-100, 15-50, or 20-30 contiguous nucleotides long.

30. The method of claim 25, wherein the linker sequence is 1-10 (e.g., 4-6) contiguous nucleotides long.

31. The method of claim 25 or 30, wherein the linker sequence is a homopolymer sequence.

32. The method of claim 31, wherein the homopolymer is poly T.

33. The method of claim 25, wherein first label is a fluorophore and the second label is a quencher.

34. The method of claim 25, wherein the first label is a quencher and the second label is a fluorophore.

35. The method of claim 25, wherein the first label is a donor fluorophore and the second label is an acceptor fluorophore.

36. The method of claim 25, wherein first label is an acceptor fluorophore and the second label is a donor fluorophore.

37. The method of claim 25, wherein the plurality of partitions is at least 1000 partitions.

38. The method of claim 25, wherein the partitions are flow cells.

39. The method of claim 25, further comprising contacting the switch domain/aptamer sequence combination that functions as a molecular switch to the target molecule and measuring a change detectable signal between the presence and absence of the target molecule.

40. A method for enriching for molecular switches, the method comprising,
(a) providing a plurality of a test nucleic acids having a 3′ and a 5′ end, wherein the test nucleic acid comprises (i) a random sequence and (i) a double stranded stem sequence comprising a double-stranded recognition sequence for a sequence-specific endonuclease and primer binding sequences that include at least part of the double-stranded recognition sequence or is closer to the 3′ and 5′ ends than the double-stranded recognition sequence;
(b) contacting the test nucleic acids with a target molecule and the endonuclease, wherein the endonuclease cleaves the double stranded stem sequence unless the target molecule triggers a conformational shift in the test nucleic acids to cause the stem sequence to disrupt the double stranded stem sequence; and
(c) selectively amplifying intact nucleic acids with primers that anneal to the primer binding sequences, thereby enriching for molecular switches that change conformation in the presence of the target molecule.

41. The method of claim 40, further comprising contacting selective amplified intact nucleic acids, or a target-binding portion thereof, with the target molecule and measuring for a change of conformation of the amplified intact nucleic acids in response to binding of the target molecule.

42. The method of claim 40, wherein one or more nucleotides at the 3′ and 5′ ends are not complementary such that the 3′ and 5′ ends do not anneal.

43. The method of claim 42, wherein the 3′ and 5′ ends each comprise at least 4-10 nucleotides that do not anneal.

44. The method of claim 40, wherein the test nucleic acids further comprise a linker sequence between the random sequence and the 3′ end.

45. The method of claim 44, wherein the random sequence is 10-50 (e.g., 20-40, e.g., 25-35) nucleotides long.

46. The method of claim 44, wherein the linker sequence is 3′ from the random sequence.

47. The method of claim 46, wherein the linker sequence is a homopolymer.

48. The method of claim 46, wherein the homopolymer is a poly T sequence.

49. The method of claim 48, wherein the linker sequence is 1-10 (e.g., 4-6) nucleotides long.

50. The method of claim 40, wherein the double stranded stem sequence is 10-14 nucleotides long with nucleotides on either end being non-complementary.

51. The method of claim 40, wherein the double stranded stem sequence is nucleotides long with nucleotides on either end being non-complementary.

52. The method of claim 40, further comprising after the providing and before the contacting, enriching the plurality for nucleic acids that form the double stranded stem sequence.

53. The method of claim 52, wherein the 3′ end of the test nucleic acids comprises one strand of a second restriction enzyme recognition sequence, and
the enriching comprises:
(i) contacting the plurality of test nucleic acids with a single-stranded oligonucleotide comprising the reverse complement of the second restriction enzyme recognition sequence, wherein if the double stranded stem sequence is present then the one strand is not available to anneal to the single-stranded oligonucleotide and if the double stranded stem sequence is not present the one strand and the single-stranded oligonucleotide anneal for form the second restriction enzyme recognition sequence; and
(ii) contacting the plurality of test nucleic acids from (i) with the second restriction enzyme, thereby cleaving nucleic acids in which the one strand and the single-stranded oligonucleotide anneal, thereby enriching for nucleic acids that form a stem loop.

54. The method of claim 53, wherein the second restriction enzyme is DdeI.

55. The method of claim 40, further comprising
(d) contacting the amplified intact nucleic acids stem loop nucleic acids with the target molecule and the endonuclease, wherein the endonuclease cleaves the double stranded stem sequence unless the target molecule triggers a conformational shift in the stem loop nucleic acids to cause the stem sequence to disrupt the double stranded stem sequence; and
(e) selectively amplifying intact nucleic acids with primers that anneal to the primer binding sequences, thereby further selecting for molecular switches that change conformation in the presence of the target molecule, wherein steps (d) and (e) are optionally repeated 1, 2, 3, 4, 5 or more times to further enrich for molecular switches that change conformation in the presence of the target molecule.


SEQUENCE LISTING (36 sequences)

SEQ ID NO: 1 - DNA, 108 bp, synthetic construct
  source 1..108: mol_type = other DNA
tcgtcggcagcgtcagatgtgtataagagacagtttttttttttttttttttttttttnnnnnnnnnntctcagctgtctcttatacacatctccgagcccacgagac

SEQ ID NO: 2 - DNA, 40 bp, synthetic construct
  source 1..40: mol_type = other DNA
gtctcgtgggctcggagatgtgtataagagacagctgaga

SEQ ID NO: 3 - DNA, 63 bp, synthetic construct
  source 1..63: mol_type = other DNA
  misc_feature 1: note = Cytosin is linked with Iowa Black FQ quencher
cacctgggggagtattgcggaggaaggtttctgtctcttatacacatctgacgctgccgacga

SEQ ID NO: 4 - DNA, 66 bp, synthetic construct
  source 1..66: mol_type = other DNA
  misc_feature 1: note = Cytosin is linked with Iowa Black FQ quencher
  modified_base order(5,7..8,12,15,19,24,26): mod_base = OTHER; note = Boronic acid-modified U base
cccgtgttagatagtaagtgcaatctcggctttctgtctcttatacacatctgacgctgccgacga

SEQ ID NO: 5 - DNA, 70 bp, synthetic construct
  source 1..70: mol_type = other DNA
ttttttttttttttttttttnnnnnnnnnntctcagctgtctcttatacacatctccgagcccacgagac

SEQ ID NO: 6 - DNA, 101 bp, synthetic construct
  source 1..101: mol_type = other DNA
tcgtcggcagcgtcagatgtgtataagagacagaaccaggagcaggccaaaaagcgcgcaagaattcctgtctcttatacacatctccgagcccacgagac

SEQ ID NO: 7 - DNA, 28 bp, synthetic construct
  source 1..28: mol_type = other DNA
ttgcgcgctttttggcctgctcctggtt

SEQ ID NO: 8 - DNA, 28 bp, synthetic construct
  source 1..28: mol_type = other DNA
ttgcgcgctttttggcctgctcctggtt

SEQ ID NO: 9 - DNA, 70 bp, synthetic construct
  source 1..70: mol_type = other DNA
tcgtcggcagcgtcagatgtgtataagagacagtttttttttttttttttttttttttcgacgcgtaatc

SEQ ID NO: 10 - DNA, 70 bp, synthetic construct
  source 1..70: mol_type = other DNA
tcgtcggcagcgtcagatgtgtataagagacagtttttttttttttttttttttttttactccagcgatc

SEQ ID NO: 11 - DNA, 70 bp, synthetic construct
  source 1..70: mol_type = other DNA
tcgtcggcagcgtcagatgtgtataagagacagtttttttttttttttttttttttttcgtagctcactc

SEQ ID NO: 12 - DNA, 70 bp, synthetic construct
  source 1..70: mol_type = other DNA
tcgtcggcagcgtcagatgtgtataagagacagtttttttttttttttttttttttttcgcatccaggtc

SEQ ID NO: 13 - DNA, 70 bp, synthetic construct
  source 1..70: mol_type = other DNA
tcgtcggcagcgtcagatgtgtataagagacagtttttttttttttttttttttttttgagcaatatatc

SEQ ID NO: 14 - DNA, 69 bp, synthetic construct
  source 1..69: mol_type = other DNA
tcgtcggcagcgtcagatgtgtataagagacagtttttttttttttttttttttttttatgcgctactc

SEQ ID NO: 15 - DNA, 70 bp, synthetic construct
  source 1..70: mol_type = other DNA
tcgtcggcagcgtcagatgtgtataagagacagtttttttttttttttttttttttttccgggggctatc

SEQ ID NO: 16 - DNA, 70 bp, synthetic construct
  source 1..70: mol_type = other DNA
tcgtcggcagcgtcagatgtgtataagagacagtttttttttttttttttttttttttccgggggcgatc

SEQ ID NO: 17 - DNA, 94 bp, synthetic construct
  source 1..94: mol_type = other DNA
gcgcataccagcttattcaattnnnnnnnnnnnnnnnnnnnnnnnnnnnnnnnnnnnnnnnnnnnnnnnnnnagatagtaagtgcaatctcggc

SEQ ID NO: 18 - DNA, 22 bp, synthetic construct
  source 1..22: mol_type = other DNA
gcgcataccagcttattcaatt

SEQ ID NO: 19 - DNA, 22 bp, synthetic construct
  source 1..22: mol_type = other DNA
gccgagattgcacttactatct

SEQ ID NO: 20 - DNA, 22 bp, synthetic construct
  source 1..22: mol_type = other DNA
gcgcataccagcttattcaatt

SEQ ID NO: 21 - DNA, 22 bp, synthetic construct
  source 1..22: mol_type = other DNA
aattgaataagctggtatgcgc

SEQ ID NO: 22 - DNA, 22 bp, synthetic construct
  source 1..22: mol_type = other DNA
gccgagattgcacttactatct

SEQ ID NO: 23 - DNA, 22 bp, synthetic construct
  source 1..22: mol_type = other DNA
aattgaataagctggtatgcgc

SEQ ID NO: 24 - DNA, 94 bp, synthetic construct
  source 1..94: mol_type = other DNA
gcgcattaccagcttattcaattgtatccttagtcccaatgtctcctggcaactcgcgcagtgccccgtgttagatagtaagtgcaatctcggc

SEQ ID NO: 25 - DNA, 30 bp, synthetic construct
  source 1..30: mol_type = other DNA
cccgtgttagatagtaagtgcaatctcggc

SEQ ID NO: 26 - DNA, 70 bp, synthetic construct
  source 1..70: mol_type = other DNA
tcgtcggcagcgtcagatgtgtataagagacagttttttttttttttttttttttttttagggcaggctc

SEQ ID NO: 27 - DNA, 70 bp, synthetic construct
  source 1..70: mol_type = other DNA
tcgtcggcagcgtcagatgtgtataagagacagtttttttttttttttttttttttttcaggacaggctc

SEQ ID NO: 28 - DNA, 70 bp, synthetic construct
  source 1..70: mol_type = other DNA
tcgtcggcagcgtcagatgtgtataagagacagttttttttttttttttttttttttttaccaggagctc

SEQ ID NO: 29 - DNA, 70 bp, synthetic construct
  source 1..70: mol_type = other DNA
tcgtcggcagcgtcagatgtgtataagagacagtttttttttttttttttttttttttaccaggagcttc

SEQ ID NO: 30 - DNA, 59 bp, synthetic construct
  source 1..59: mol_type = other DNA
agtggatccagccggaggctggattagagactgaggagctagtttttgctggatccact

SEQ ID NO: 31 - DNA, 60 bp, synthetic construct
  source 1..60: mol_type = other DNA
agtggatccagccggaggctggattagagactgaaggagctagtttttgctggatccact

SEQ ID NO: 32 - DNA, 60 bp, synthetic construct
  source 1..60: mol_type = other DNA
agtggatccagcggagctggataagagagagggatacatgcaatttttgctggatccact

SEQ ID NO: 33 - DNA, 60 bp, synthetic construct
  source 1..60: mol_type = other DNA
agtggatccagcggagctgggataagagagaggatacatgcaatttttgctggatccact

SEQ ID NO: 34 - DNA, 60 bp, synthetic construct
  source 1..60: mol_type = other DNA
agtggatccagccggaggctggattaagagactgaagagctagtttttgctggatccact

SEQ ID NO: 35 - DNA, 59 bp, synthetic construct
  source 1..59: mol_type = other DNA
agtggatccagccggaggctggattagagactgaggggctagtttttgctggatccact

SEQ ID NO: 36 - DNA, 59 bp, synthetic construct
  source 1..59: mol_type = other DNA
agtggatccagcggagctggataagagacaggatacatgcaatttttgctggatccact